(19) United States
(12) Patent Application Publication
(10) Pub. No.: US 2023/0166256 A1
(43) Pub. Date: Jun. 1, 2023

(54) METHODS AND SYSTEMS FOR SAMPLE PROCESSING UTILIZING FILTER AID MATERIALS AND AGGREGATING SAMPLERS FOR EQUIPMENT

(71) Applicant: Fremonta Corporation, Fremont, CA (US)

(72) Inventors: Yongqing Huang, Newark, CA (US); Florence Q. Wu, Milpitas, CA (US); Eric C Wilhelmsen, Milpitas, CA (US); Garth F Hoffmann, El Dorado Hills, CA (US)

(21) Appl. No.: 17/956,308

(22) Filed: Sep. 29, 2022

Related U.S. Application Data
(60) Provisional application No. 63/249,787, filed on Sep. 29, 2021.
(60) Provisional application No. 63/343,027, filed on May 17, 2022.

(51) Int. Cl. B01L 3/00; B01L 7/00; C12M 1/00

(52) U.S. Cl. CPC B01L 3/502753; B01L 7/52; B01L 3/502715; C12M 47/06; B01L 2300/0681; B01L 2300/087; B01L 2200/10; B01L 2400/0487; B01L 2400/0644

(57) ABSTRACT

Methods and apparatus for improved microbial sampling of foods and sample treatment are provided herein. Such methods may include sampling production lots of produce or other food items such as meat using an aggregating sampler to create one or more samples. Methods and devices that improve concentration of the fluid sample obtained from the aggregate sample include filtering of fluid sample through a filter and/or filter aid materials and lysing target material trapped within the filter and/or filter aid materials to release molecules from targeted microorganisms, which are recovered in a concentrated fluid sample for testing. Sample treatment systems and methods can be automated with various buffer reservoirs and removable cartridges that facilitate controlled flow of fluid sample therethrough to produce a purified, concentrated fluid sample, typically within two hours or less. Such systems can further be configured with removable cartridges and use with sample filter cups and collection cups.

### REFERENCES TO RELATED APPLICATIONS

This application is a Non-Provisional application of and claims the benefit of priority of U.S. Provisional Application Nos. 63/343,027 filed May 17, 2022 and 63/249,787 filed Sep. 29, 2021, each of which is incorporated herein by reference in its entirety.

### BACKGROUND

### Field of the Disclosure

The present disclosure relates generally to improving the food safety of ready-to-eat produce and other food items and providing process validation, and more particularly, to methods and apparatus for microbial sampling of food items and other materials.

### Description of Related Art

The microbial testing process has undergone tremendous change in recent years. Traditional plating techniques for enumeration and detection have given way to faster and more specific antibody and molecular techniques. These newer techniques may not require time for colonies to form but they may generally require cultural enrichment to increase the concentration of the target organism and dilute interferences.

In the ready-to-eat produce industry, millions of dollars are spent collecting grab samples attempting to demonstrate the safety of products in an effort to meet demands by customers for an ever-increasing numbers of tests. These efforts may be technically and statistically flawed and may not meet the expectations of assuring food safety. Particularly, grab samples are too small to represent the production lots of material. Production lots of material are too heterogeneous for grab sampling to be descriptive of the production lot. Further, results arrive too slowly to make decisions without sacrificing quality. Pathogens levels are generally so low that the occasional positive sample reflects the background that is always present rather than a deviation from the norm. The ready-to-eat produce industry may benefit from an effective assay of cross contamination and cross contamination control. It may also benefit from an effective measure of process efficacy and deviation in processing. Increasing the effectiveness of raw material testing may help improve food safety practice.

Thus, as the demand for microbial sampling continues to increase, there exists a desire for further improvements in sampling techniques and technology. Preferably, these improvements should be applicable to other related technologies and the methods and devices that employ these technologies.

### BRIEF SUMMARY

The systems, methods, and devices of the disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure provide advantages that include improved microbial sampling of foods and other products.

Certain aspects provide a method for microbial sampling of foods and other products. The method generally includes gathering a microbial sampling from one or more items to test for one or more targeted microorganism, extracting the microorganisms from the microbial sampling, concentrating the microorganisms, and testing. Concentrating the microorganisms can include filtering a fluid sample through a filter and/or filter aid materials, and then processing the target material trapped within the filter and/or filter aid materials, such as by lysing, to release molecules from the targeted microorganisms and recovering the molecules for testing. In some embodiments, the filter includes a filter membrane and any target material trapped within the filter is lysed to release molecules of the target for subsequent recovery and testing. In some embodiments, the filter includes filter aid materials and any target trapped within the filter aid materials is lysed to release molecules of the target for subsequent recovery and testing. In some embodiments, the filter includes a filter membrane and filter aid materials and any target trapped within the filter membrane and/or the filter aid materials is lysed to release molecules of the target for subsequent recovery and testing.

Certain aspects provide a method for microbial sampling of foods. The method generally includes providing at least one aggregating sampler at one or more sampling locations, and sampling, using at least one aggregating sampler, a production lot of produce creating one or more samples that makes up a microbial sampling. In some embodiments, the aggregating sampler includes a sample medium, which can be any suitable size and shape. The sample medium can be a non-woven cloth (e.g. MicroTally cloth), typically a synthetic polypropylene fabric or any suitable material. In some embodiments, the sample medium cloth has a central opening (e.g. slit or hole) that fits atop an apex of a conical hat of a vertical filling machine so as to releasably attach to the conical hat and obtain an aggregate sample of produce dropped onto the sample medium without requiring additional separate components.

Aspects generally include methods, apparatus, systems, computer readable mediums, and processing systems, as substantially described herein with reference to and as illustrated by the accompanying drawings.

In another aspect, the invention pertains to sample treatment system and associated methods of treating a sample. Such treatment systems can include a flow through network of various treatment regions that produce a purified, concentrated sample without mechanical transfer of sample and without requiring any enrichment, typically within two hours or less. In some embodiments, the fluid sample can be subjected to enrichment before, concurrent with, or after concentration by filtration. In some embodiments, the system includes an extraction bag, a filtration module, a mixing/binding vessel, and a nucleic acid affinity column. In some embodiments, the extraction bag is connected to an extraction buffer reservoir, the filtration unit is connected to an in situ lysis buffer reservoir, the mixing/binding reservoir is connected to a binding buffer reservoir and the affinity column is connected to a washer buffer reservoir and elution buffer reservoir. Each can further be attached to a waste reservoir. In some embodiments, the system is configured to allow flow of fluid sample through the network of conduits by use of actuatable valves and one or more vents. The system can include a single vacuum source to draw fluid through the network. The system can include a removable cartridge having the filtration module, mixing/binding vessel and affinity column thereon and a removable sample injection unit that facilitates transport of fluid sample from the extraction bag to the cartridge. In some embodiments, the injection unit and cartridge include fluidic tubing that are actuated by pinch valves of a system interface.

In one aspect, the invention pertains to a method of treating a sample by use of a sample filter cup. Such methods can include steps of: filtering a fluid sample with a sample cup having filter aid materials therein, thereby trapping target molecules from the fluid sample in the filter aid materials and discarding waste water from filtering the fluid sample; placing the sample cup having the filter aid materials in a sample treatment system; fluidically coupling the sample cup with a binding/mixing vessel, nucleic acid affinity column, and associated buffer reservoirs and a waste receptacle of the system, wherein the system further includes a network of fluidic conduits and one or more valves that facilitate controlled fluid flow through the system; and operating the system by use of one or more vacuum sources and actuation of the one or more valves so as to control facilitate controlled flow of the sample and various buffers through the system. Operation of the system performs the various steps needed to prepare and concentrate the fluid sample for testing without requiring any enrichment. In some embodiments, the fluid sample can be enriched before, concurrent with, or after concentration by filtration with the filter aid materials. In some embodiments, enrichment media is added to the filter aid materials to enrich concurrent with filtration. In some embodiments, these steps performed by the system include: lysing the target molecules from the filter aid materials disposed in the sample cup by use of an in situ lysate buffer introduced into the sample cup by the system so as to create a lysed fluid sample having DNA released from the target molecules, transporting the lysed fluid sample containing the released DNA, by application of vacuum, from the sample cup into the binding/mixing vessel and mixing with washing and/or binding buffers; transporting, by selective actuation of the one or more valves, the mixed fluid sample containing the released DNA into a nucleic acid affinity column and adding an elution buffer, and eluting, by selective actuation of the one or more valves, a concentrated fluid sample containing the DNA from the target molecules from the column into a test tube. In some embodiments, at least a portion of the system including the binding/mixing vessel and the nucleic acid affinity column is configured as a removable/replaceable cartridge, that fits into a larger system having the buffer reservoirs and vacuum source. In some embodiments, the method includes use of a standard, off-the-shelf sample cup.

In another aspect, the invention pertains to a sample treatment system that includes a receptacle or interface for receiving a sample filter cup having filter aid materials therein with target molecules from a fluid sample filtered therethrough; an inlet configured for introducing an in situ lysate buffer into the sample cup when disposed within the system; a binding/mixing vessel fluidically coupled with the sample cup and having an inlet configured for transport of washing and/or binding buffers therein; a nucleic acid affinity column fluidically coupled with the sample cup and having an inlet configured for transport of elution buffer therein; and a network of fluidic conduits and one or more valves that facilitate controlled fluid flow through the system by application of a vacuum source through a vacuum port so as to draw lysed fluid sample from the sample cup into the binding/mixing vessel, draw a mixed fluid sample containing released DNA into the column and elute a concentrated fluid sample containing the released DNA into a test tube. In some embodiments, the system, or at least a portion thereof, is configured as a removable/replaceable cartridge configured to be fluidically coupled within a larger system having multiple reservoirs for the various buffers. In some embodiments, the system is configured for use with a sample cup that is a standard, off-the-shelf sample cup.

In still another aspect, the invention pertains to a sample treatment system for treating a sample that includes: a filter cup having a filter and/or filter aid materials therein for filtering and concentrating a fluid sample; a binding/mixing vessel fluidically coupled to one or more buffer reservoirs; a nucleic acid affinity column fluidically coupled to one or more additional buffer reservoirs; and a collection cup having a sample vial for collection of purified, concentrated fluid sample from the nucleic acid affinity column. The filter cup, the binding/mixing vessel, the affinity column and the collection cup can be fluidically interconnected so as to facilitate controlled flow of the fluid sample through the system and into the sample vial upon selective application of vacuum from a vacuum source. In some embodiments, the one or more buffer reservoirs comprise a neutralizing buffer and/or binding buffer and the one or more additional buffer reservoirs include a washing buffer and/or an elution buffer. The filter cup can be readily removable from the system and includes filter aid materials for purifying and concentrating the fluid sample therein. A lysis buffer can be added to the filter cup for lysing the fluid sample in situ within the filter aid materials. In some embodiments, the system includes a heater above the filter cup. In some embodiments, the respective elements are interconnected within a network of fluid conduits having one or more valves to facilitate controlled fluid flow through the respective elements to produce the purified and concentrated sample. One or more air tubes are connected with the vacuum source for drawing a vacuum in the binding/mixing vessel and/or the collection cup. The system can further include a control unit configured to automate, at least partly, the actuation of one or more valves and the vacuum source so that the fluid sample flows through the system in at least a party automated manner. In some embodiments, the collection cup includes container and a lid that seals atop the container, where the lid is configured to support the nucleic acid affinity column such that that the affinity column empties into the container. In some embodiments, the lid further includes a vacuum port for connecting with an air tube in communication with the vacuum source for drawing a vacuum within the collection cup. The lid can further include a plug that seals atop the affinity column, the plug having one or more conduits defined therethrough for passage of the fluid sample, a washer buffer and an elution buffer. In some embodiments, the collection cup includes a vial stand within for supporting the sample vial beneath the affinity column within a container of the cup. The affinity column and sample vial are readily removable from the collection cup, and the collection cup is readily removable from the system.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 illustrates an example process workflow of sample processing utilizing filter aid materials to concentrate a target analyte within the fluid sample, in accordance with some embodiments of the invention.

FIG. 2 illustrates an example method of sample processing utilizing filter aid materials, in accordance with some embodiments.

FIG. 3 illustrates an example method of sample processing utilizing filter aid materials, in accordance with some embodiments.

FIG. 4 illustrates an example method of sampling and testing for a target analyte employing sample processing utilizing filter aid materials, in accordance with some embodiments.

FIG. 5 illustrates an aggregating sampler, in accordance with some embodiments.

FIG. 6 illustrates an aggregating sampler shaped as a glove for manual sampling, in accordance with some embodiments.

FIG. 7 illustrates an aggregating sampler that includes a heated glove for manual sampling of frozen food products, in accordance with some embodiments.

FIG. 8 illustrates an aggregating sampler that includes a stationary sampler disposed along a food product conveyor, in accordance with some embodiments.

FIG. 9 illustrates an aggregate sample that can be used in sample processing utilizing filter aid materials, in accordance with some embodiments.

FIG. 10 illustrates a filter device that can be used in sample processing utilizing filter aid materials, in accordance with some embodiments.

FIG. 11 illustrates a vertical form filling and sealing system for fresh produce and an associated aggregating sampler, in accordance with some embodiments.

FIG. 12A-12E illustrates aggregating sampler for placement atop a conical hat in a vertical filling machine, in accordance with some embodiments.

FIG. 13A-13C illustrates attachment mechanisms for aggregating samplers for mounting on equipment, in accordance with some embodiments.

FIG. 14-15 illustrates schematics of sample treatment systems, in accordance with some embodiments.

FIG. 16 illustrates an example sample treatment system having a removable cartridge, in accordance with some embodiments.

FIG. 17 illustrates a removable sample injection unit for the treatment system, in accordance with some embodiments.

FIG. 18 illustrates a removable sample treatment cartridge for the treatment system, in accordance with some embodiments.

FIG. 19 illustrates a sample treatment system for use with a sample cup having filter aid materials, in accordance with some embodiments.

FIGS. 20-21 depict example methods of treating a sample by use of a treatment system, in accordance with some embodiments.

FIGS. 22-23C depict an exemplary flow-through sample treatment system, in accordance with some embodiments.

FIGS. 24A-24B depict an exemplary collection cup and FIGS. 25A-25C depict a tool for manufacturing a plug for the affinity column, in accordance with some embodiments.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Aspects of the present disclosure provide apparatus, methods, and/or systems for automated and semi-automated microbial sampling of foods and other materials. Other materials can be as diverse as water or air streams. More commonly, it will include kindred products of food such as pet food, medical materials or dietary supplements where microbial testing is needed to confirm hygienic operation. Sampling can be active as mediated by material flow or operator mediated by applying the sampler to a surface. Sampling can also be more passive and depend on passive contact or gravity sedimentation.

The following description provides examples, and is not limiting of the scope, applicability, or examples set forth in the claims. Changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim. The word “exemplary” is used herein to mean “serving as an example, instance, or illustration.” Any aspect described herein as “exemplary” is not necessarily to be construed as preferred or advantageous over other aspects.

In one embodiment, devices are assembled and linked to a rapid reporting system to provide a more representative sampling and faster analysis of a food product or other material. In another embodiment, a similar system provides for robotically sampling a field crop and delivering results with a mobile laboratory. Both embodiments can include a two-stage screening system for speed and for economy, but a conventional testing approach can be considered to take advantage of the sampling improvements.

There has been little evolution in sampling and sample preparation for submission to these advanced and rapid analytical techniques. One technique is to collect periodic samples or random grabs from a lot. This sample is extracted by homogenization or stomaching and then either a portion analyzed directly when high populations are expected or enriched prior to analysis. Liquid samples and particularly water samples can be filtered to allow analysis of larger samples. For special purposes but generally not for routine analysis liquid samples can be concentrated by centrifugation to pellet microorganisms. There are statistically based sampling plans as recommended by academics or international organizations, but these sampling plans are rarely practical and mostly cost prohibitive. Such sampling plans in use based on periodic samples or random grabs have a very low data density. Furthermore, these sampling plans intrinsically have sampling biases due to lot geometry where portions of the lot are essentially not sampled and the heterogeneous nature of microorganism distribution.

Some microbial testing may assume that the test organisms are evenly distributed allowing a grab sample to be representative of the whole. This flawed assumption only accounts for inhomogeneous distributions in aggregate and requires many samples to characterize the microbial population of a lot. Test and release inspection based on grab samples may be flawed to the extent that grab sampling can inherently miss significant contamination. Furthermore, the size the sample limits the detection limit to levels that may be order of magnitude above the background rate and the level where risk becomes imminent. On the other hand, microbial proliferation during enrichment culture occurs under conditions selected to favor the growth of the potential pathogens which often have no relationship with the commercial conditions of storage and therefore generates extorted perceptions of the microbial growth risk.

One or more embodiments as disclosed herein may address these short comings of the current sampling and testing practice and provides a system to generate more meaningful, real time or near real time, onsite assessment of microbial contamination risk.

Example of Automated and Semi-Automated Microbial Sampling of Foods and Other Materials

In accordance with one or more aspects of embodiments disclosed herein, automated and semi-automated microbial sampling of foods and other materials is provided. For example, devices may be assembled and linked to a rapid reporting system to provide a more representative sampling and faster analysis of a food product. In another case, a system may provide robotic sampling of a field crop and may deliver results with a mobile laboratory. Both examples may include a two-stage screening system that may provide speed and for economy. Although less advantageous, it is reasonable to take advantage of improved sampling with conventional enrichment and detection systems.

In one or more cases, a method and system of microbial sampling includes providing a sampling sheet, such as a MicroTally® Sheet, which is then used with a continuous sampling device (CSD) to collect biological agents. The CSD may change the sampling sheet automatically between samples. The sampling sheet is then used for analysis. Particularly, target bacteria may be removed from the sampling medium. The samples of target bacteria are cleaned up and concentrated using a treater and made suitable for analysis. The treated sample is then analyzed using molecular or biochemical methods and target agents are detected with acceptable accuracy and sensitivity. A cloud based data reporting system with user appropriate dashboards to report actionable information and facilitate timely decision making may also be provided.

For example, specific operations for microbial sampling can begin with gathering a microbial sampling from one or more food items which may include, for example, produce, meat, and other food products or other materials. Operations can include extracting microorganisms from the microbial sampling, and concentrating the microorganisms. Operations can further include cleaning the microorganisms. Further, the operations can include tallying the relative presence of the microorganisms and any potential pathogens, aggregating this information of a microorganism tally from the tallying of microorganisms into a microorganism report, confirming the microorganism tally, and reporting the microorganism report of the microorganism tally.

In one or more cases, sampling may include using a sampling sheet or swab that collects a sample and is stomached in a 200˜300 micron partitioned bag with 200 mls of suspension buffer and stomached, although volume can range lower (e.g. 150 mls). Concentration and cleanup of the sample may include siphoning a suspension buffer and entrained organisms through proprietary sequential filter and the targets may be deposited on a 0.22 or a 0.45 micron PC or MCE filter. The targets are analyzed from the filter which may include for example, target cell lysis, nucleic acids (NAs, include DNA, RNA or both) extracted, NAs purified and qPCR or Loop-Mediated Isothermal Amplification (LAMP) being run for Index elements including pathogen intensity, enteric status, and/or positive control. Cloud based reporting based on, for example Ignition and SQL database, may be provided with user appropriate dashboards to provide timely actionable information

In accordance with one or more cases, pathogens may be confirmed by resampling and enrichment procedures, by resampling and doing definitive pathogen tests, or by doing confirmation tests on the original NAs depending on the regulatory guidance. In one or more cases a cassette and cartridge system may be implemented to streamline the sampling process. Although not required, eliminating the use of a stomacher and partitioned bag may be provided in one or more cases. A binding collector may be provided to replace the PC or MCE membrane filter which may help streamline the sample delivery to the detector and potentially eliminate the need for NAs purification. Use of Ribosomal RNA may become a new standard for one or more such cases. In one or more cases, a detector may use flow amplification and laboratory on a chip type technology to further reduce detection times and costs.

To best address all the short comings in current practice numerous improved elements may come together. Taken individually each improvement addresses some of the short comings and yields some advantages. Leaping all the way to a complete solution may beyond the sophistication of some industry classes so intermediate steps are considered for each element. For the initial discussion, the elements under consideration include: Sampling, Extraction, Concentration, Cleaning, Screening Detection, Second Stage Sampling of Suspected Lots, Reporting, and Information Roll Up. Although not as desirable, a more conventional enrichment can be used instead of concentration. After such enrichment, any number of detection systems can be used to detect the presence or absence of a target organism. Each element is discussed below.

One fundamental improvement pertains to methods of concentrating the fluid sample. While filtering of fluid samples is common practice, the filtering process removes some of the targeted microorganism (i.e., target analyte), such that the amount remaining in the filtered fluid sample might fall below detection limits. The target analyte remains bound or trapped within the filtering material and typically cannot be removed by conventional washing techniques. By lysing the trapped material trapped within the filter material, such as by chemical, mechanical, ultrasonic, or thermal means, molecules (e.g. DNA, RNA, etc) of the target analyte can be released from the target analyte that remains trapped or bound to the filter material. These released molecules can then be recovered by various approaches (e.g. washing, centrifugation, filtering, etc.), so that the resulting fluid sample has a higher concentration of molecules of the target analyte than would otherwise be possible, thereby improving detection of the target from the sample. This approach is particularly advantageous for testing of aggregate samples that might contain lower levels of the target analyte, and is further advantageous as it allows for more rapid testing without requiring enrichment.

Thus, the timeframe from sampling to information may be driven by the needs of the business class but is not limited thereto. Short shelf-life product can justify greater speed. Valuable commodities such as meat products will want more testing to limit exposure when a problem is expected or anticipated. These factors will impact the degree to which a complete automated solution is implemented or conversely when a system more akin to current practices is used to gain some of the benefits of improved sampling.

Sample Processing Workflow

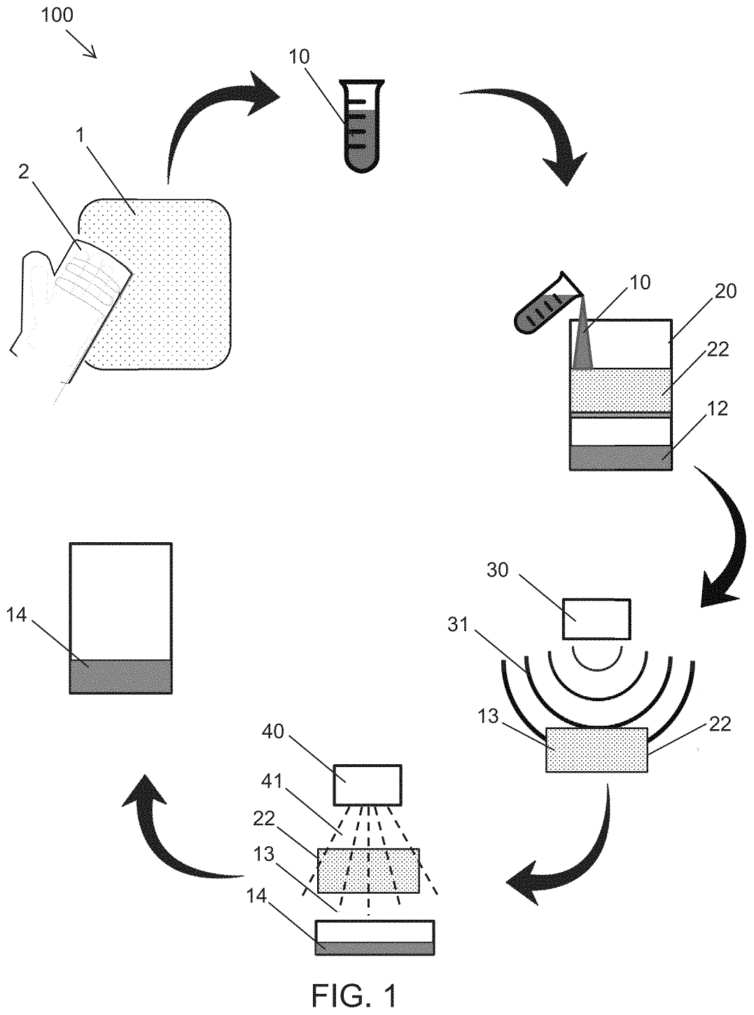

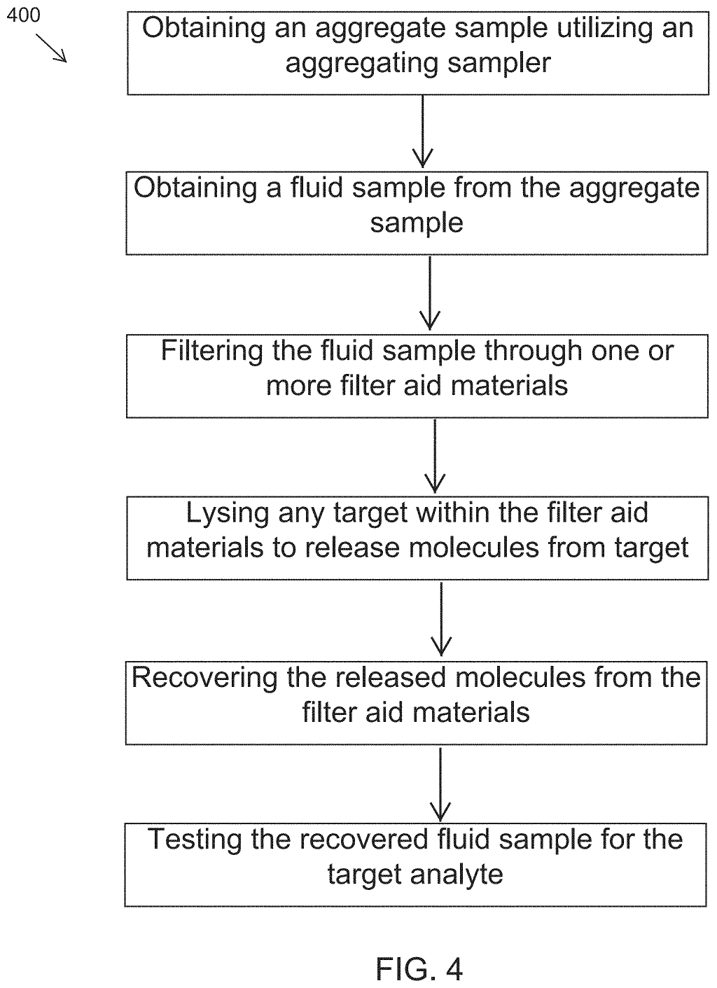

An exemplary workflow of sample processing with improved concentration by utilizing filter aid materials is depicted in FIG. 1. Initially, a food product 1 is sampled by sampling medium 2, in accordance with any suitable sampling protocol, including the aggregate sampling methods described herein. While a heated glove samplers is depicted here, it is appreciated that the sample can be obtained from any type of sampler. From this sample, a fluid sample 10 is obtained. As described herein, various known methods may be used to obtain the fluid sample from the sample medium (e.g. cleaning, washing, extraction, etc). In some embodiments, no enrichment is required. The fluid sample 10 is then concentrated by filtering the fluid sample through filter 20, which includes filter aid material(s) 22 in which the target molecules are trapped, and producing the remaining fluid waste product 12, which can be discarded. However, as noted above, a significant portion of the target analyte, if present, may remain trapped within the filter aid material(s). Typically, most or all of the target molecules are trapped in the filter and filter aid material. Thus, the lysing means 30 is applied to the filter aid material(s) 22, which lyses 31 the target material trapped within the filter aid materials so as to release molecules 13 from the target analyte trapped within the filter aid material. The lysing means 30 may include but is not limited to: introducing enzymes or chemicals; applying mechanical energy; applying ultrasonic energy; applying thermal energy; or any combination thereof. Next, a recovery means 40 is used to recover 41 the released molecules 13 within concentrated fluid sample 14. The recovery means can include but is not limited to any of: washing, filtering, centrifugation, precipitate separation, binding and elution or any combination thereof. This concentrated fluid sample 14 can then be processed, if needed, and tested for the target analyte, which provides a more accurate count of the target analyte from the original fluid sample 10. The filter aid material(s) can be lysed and processed, in situ, within the same filter, or can be removed and processed separately. FIG. 4 depicts this general workflow as method 400.

Examples of Sampling

There are practical limits to the amount of product that can be sampled by conventional means. Without heroic efforts samples are limited to small fraction of a pound (generally 150 grams or less but some labs are routinely testing as much as 300 grams) which lead to operating curves for c=0 acceptance that have an inflection at about 1 CFU/pound. The net effect of such sampling and testing is the erroneous belief that the worst lots are detected when 1 positive is found in many hundreds or thousands of samples. Unfortunately, this testing is so far removed from the range of interest; it is little more than a random selection of lots to be rejected.

A manual sheet-based sampling can increase the effective sampling weight 20 or 30 fold which is enough to move the operating curve about an order of magnitude to the left to about 0.1 CFU/pound if the same c=0 inspection criteria are used. Similarly, if a continuous sampling method is used the effective sample can be increased 200 to 300-fold yielding an additional order of magnitude in LOD to about 0.01 CFU/pound.

The surface area of an aggregating sampler affords advantages beyond material sampling when greater sensitive is desired for surface and fluid flow sampling. Water and air stream sampling are two examples of where flow sampling is advantageous. The surface area is also applicable to the sampling of surfaces where topical contamination is of concern.

The use of the sampler has the advantages of being nondestructive and can yield executional efficiencies. However, the real advantage comes when this LOD is traded off for statistical process control with a two-stage acceptance criterion where deviation from normal are detected as opposed to randomly selecting lots for real location. This concept is discussed more fully below when this discussion returns to screening detection. This is an important distinction when the goal is to improve the microbial safety of a product or material. This line of argument also permits the more rapid detection of many cells rather than waiting for one cell to grow into many cells. With the recognition of the power of the larger and effective sampling procedure, there may be a need to expand the range of tooling to apply this technology to a broader array of products with alternative geometries and increased levels of automation. For example, the geometry can be altered to allow sampling of a powder flow through a pipe with a circular geometry where a pipe segment is exchanged between lots. The pipe section would either be lined with sampling material or better include baffles maximizing product contact with the sampling surfaces. Alternatively, one can envision vertical chutes below pocket fillers to sample product just prior to bagging in a form fill and seal machine. Similarly it can be sampled just prior to the weigh scale by attachment to the dispersion cone.

A microbial sampler may be included, for example, non-woven fabric, various micro fiber materials, sponges, and/or any absorbent sheet material. A non-woven polypropylene or polyethylene fabric are of particular note as these materials are allowed for food contact and therefore have very low extractables which might otherwise contaminant the material stream under examination. Additionally, the utility of these sampling approaches can be increased with automation. A feed cartridge can be used to deliver multiple sheets to the sampling location at one time. This cartridge would be placed on the line after sanitation has completed all preparations protecting the drive mechanisms from the harsh cleaning process. Similarly, a magazine of cassettes can be loaded to collect individual pieces of sampling material. Both the cartridge and cassette are engineered to advance the sampling material when appropriate, (e.g. When a lot is completed, when a tote is moved, etc.). The motive force to advance sampling sheets can be provided by a motor, or supplied by a manual crank or handle, or by an operator depending on the specifics of the operation. Both cassette and cartridge may be designed to protect the microbial integrity of the contents. The cartridges may prevent microbial growth after sampling, external contamination, and cross contamination between lots. The sampling material can be mounted on an inert backing material to facilitate the sanitary placement of sampling sheets. Alternatively, sheets of sampling material can be separated by short spaces of inert material to ensure that used sampling materials do not contaminate other sheets.

The usual design parameters for process equipment may be applied to these devices including, for example, an aggregating sampler. For example, heavy gauge 316 stainless is an appropriate material. When the sampling device is not in place, the location may be passive such as a dead plate where product passes without damage or hindrance. It may be sanitary design from the beginning such that it is easily cleaned. In one case, for this automation to have maximal benefit, the cartridges may carry the information regarding the sample they contain. If the sample is used in a manual mode, an electronic transfer of this information along with the sample is also advantageous to avoid human error and speed the flow of information. This transfer of information can occur through the cloud using barcodes, a database and location information.

Another class of geometries is necessary to extend the power of this sampling to agricultural commodities in the field. Such sampling has utility beyond testing for human pathogens in that it can be used for testing for plant pathogens that can decrease the productivity of a crop. For example, early detection of mildew spores prompt early harvest of a spinach field. Detection of blight in a wheat or corn field might prompt the use of a disease control measure on the affected field before the blight destroys the crop. Depending on the field crop, an octopus-like tentacle configuration may be the appropriate geometry where strands of the sampling material are slide across the surface of the crop. These strands can be fuzzy cords or strips of material depending on what provide the greatest effective contact. These tentacles can be contacted to the crop by various mechanisms including robots, tractors, hand carrying or drones. It is most important that the altitude be held constant to allow contact while minimizing damage to the crop. To increase the effectiveness of the sampling, it can be advantageous to wick moisture down these tentacles or install vacuuming or sucking mechanism. For crops that present a more uniform top surface such as baby greens or spinach, sheet materials can be more effective as new upturned leave surfaces are missed. For these crops, air based sampling with suction or electrostatics that increases microbial sampling efficiency presents an interesting alternative.

For manual sampling, forming the sampling material into pockets, mittens or gloves can facilitate use. Ease of use will generate greater compliance with the sampling protocol. In a manual mode, the duration of contact is a factor in determining the effectiveness of sampling. Typical durations are on the scale of minutes. Two-five minute durations will work for most applications.

Various sample operations and devices for aggregating sampling can be used in accordance with the sample processing techniques described herein. Such aggregating sampling devices and operations can include any of those described herein, or those described in U.S. application Ser. Nos. 16/057,137; 16/525,350; and 63/134,671, the entire contents of which are incorporated herein by reference for all purposes.

Specifically, operations can include at least one aggregating sampler at one or more sampling locations creating one or more samples that makes up a microbial sampling. The one or more samples may be configured to be processed to indicate if pathogens are present at no greater than a normal background. The one or more sampling locations may include at least one of in a field, at harvest, just after dumping or cutting, in a wash system, or after the wash system. In one or more cases, additional operations may be include such as, for example, assessing, using the aggregating sampler, a level of cross contamination control to validate or verify a wash process. In one or more cases, an aggregating sampler may be provided that sufficiently samples a production lot of ready-to-eat produce to confirm that pathogens are present at no greater than the normal background. In one or more cases, an aggregating sampler may assess the level of cross contamination control to validate or verify a wash process.

In one or more cases, an aggregating sampler and a sampling location of the aggregating sampler may be provided. Additionally, sampling by the aggregating sampler may be provided to generate one or more desired samples. Analysis of the one or more samples and interpretation of the analysis results may also be provided. These elements can be practiced individually or together to enhance food safety.

The aggregating sampler may include a collection surface and an apparatus for holding and positioning the collection surface such that the collection surface contacts product that is to be sampled for micro-organisms or other targets. In one or more cases, a surface with a sampling efficiency that allows an increased effective sampling size when a production lot of the product is sampled may be provided. For example, two hours of production of a leafy green product may be sampled with such a device. During the two hours a large amount, for example 10,000 to 30,000 pounds, of product will have crossed the sampling surface. If the sampling device is at least 25% efficient as shown in bench scale studies, the effective sample size may be 2500 to 7500 pounds. These sample sizes are enormous when compared to the few hundred grams of a normal grab sample.

In one or more cases, there are a number of factors to consider in selecting or designing an aggregating sampler. Initially, a sampling surface that is compatible with the product is provided. This typically means that the sampling surface is a food grade material. For example, in one or more cases, a non-woven polyolefin cloth has proven to be effective. Another factor may include a design that allows safe and rapid exchange of the sampling surface. Another factor that may be included relates to any apparatus left on the line being easily cleaned and may incorporate a sanitary design. For example, food grade stainless steel may be a material selected when implementing an aggregating sampler.

For produce testing, there are a number of areas where aggregating sampling may yield improvements in food safety. For example, some locations include: 1) In the field; 2) At harvest; 3) Just after dumping or cutting; 4) In the wash system; or 5) After the wash system. In each area, there are particular locations that may be selected for aggregate sampling, but these will vary with the configuration of the specific line and the product to be sampled. Not all areas will be appropriate for all products. The selections may be guided by the desired information. Currently, grab samples are often taken in all of these areas but these grab samples are unable to represent the population under examination and such sampling is generally destructive.

According to an example, an aggregating sampler may be provide that can replace the current practice of taking a grab sample in a field by cutting leaves. Particularly, the aggregating sampler can be configured such that it can be carried by hand or mounted on a device designed for traveling through the field. The collection surface for field sampling may be divided to allow more conformity to the crop surface.

According to another example, at harvest, an aggregating sampler may be placed on the harvester such that product is sampled during the harvesting process. This approach would allow pairing of harvested product with specific information. Although not required, placement of the aggregating sampler may be just after any sorting is done in the field. For example, if rocks are sorted from the product by density classification, these rocks need not cross the sampler surface.

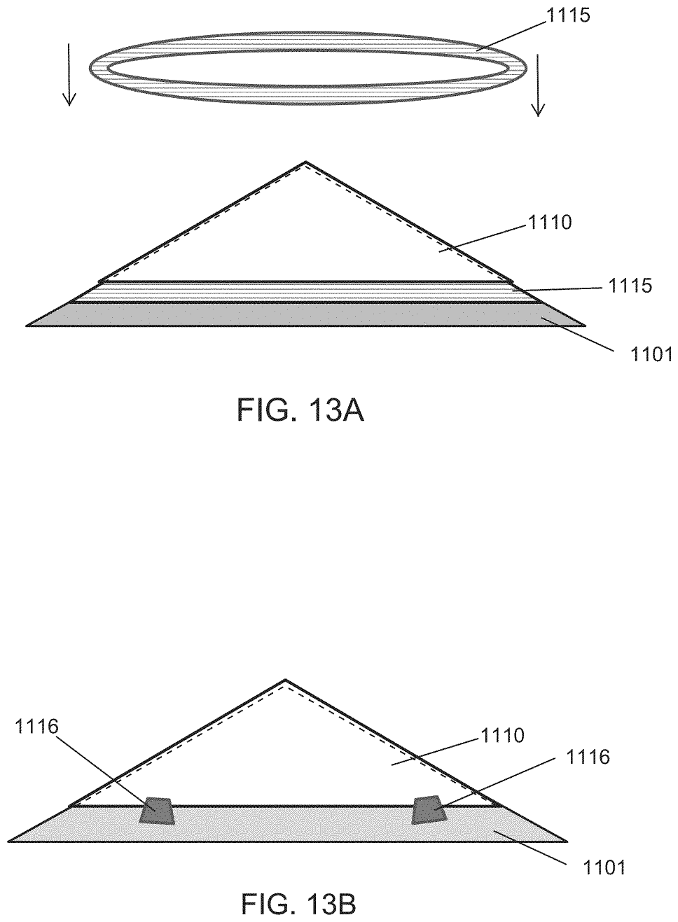

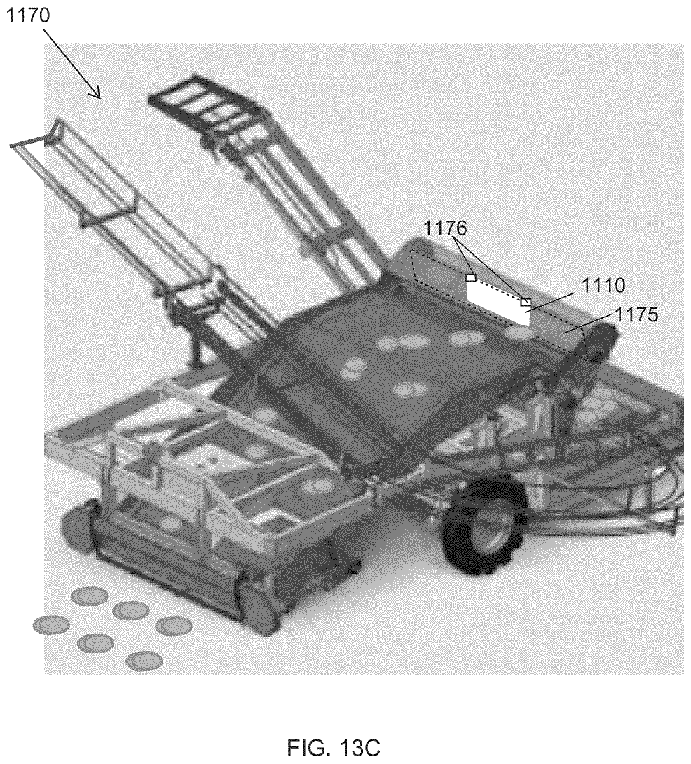

In another aspect, the sampling member can similarly be used in harvesting equipment. This is known as “at harvest sampling,” which is another special form of CSD sampling. Given the wide variety of harvesting equipment and product conveyed on this equipment, a one size fits all cassette and baseplate system would likely be impractical. Instead, a variety of clips can be used to attach the sampling member (e.g. sampling cloth, MicroTally® swabs) directly to preexisting components of harvester equipment. In some cases, the product is being ejected from the harvester where it strikes a surface designed to force the product to fall into bins. In these cases, attaching the swabs to these almost vertical surfaces can generate representative samples. For example, as shown in FIG. 13C, a sampling member 1100 can be held in place on a strike plate 1175 of a harvester 1170 by one or more clips 1176 attached to the strike plate. While a strike plate is preferred, any suitable surface contacting the product being harvested could be used. Although a leafy produce harvester is shown, it is appreciated that this approach could be suitably modified to fit a wide variety of harvesting machines and equipment, so long as the sampling member 1100 can be releasably attached to a surface of the equipment that contacts the product being harvested and conveyed. This mode of sampling can be useful for the sampling of baby leaf products which are mechanically harvested. In other cases, the product falls against surfaces which direct the product towards bins. This mode is more common for head products such as Romaine or iceberg lettuce. It is clear that this mode can be used with other food products that are conveyed in a similar manner during harvesting.

In another example, just after dumping or cutting and before washing, the product may still have the flora found in the field. An aggregating sampler placed early in the process can sample these organisms. Just after cutting or chopping, the interior of some products will be exposed for the first time allowing a more representative sample to be taken.

In a wash system, an aggregating sampler can collect a different type of sample in accordance with one or more examples. This sampler may test the cross-contamination control of the wash system. The organisms collected will reflect the two most probable mechanisms of cross contamination, water mediated cross contamination and product to product cross contamination. An aggregating sampler, placed in the flow of the conveyed product will be impinged by both the water and the product. To avoid overly hindering product flow depending on the line design, the sampler can be placed at any angle from parallel to the product flow to complete perpendicular to product flow. The sampler can also be a comb-like device with multiple collecting probes among product and in the wash flow. The angle of attachment may affect the balance between water mediated cross contamination and product to product cross contamination observed. In either case, this type of sampling may be used to validate cross contamination control and effectiveness of wash solution.

Sampling in the wash system is a case where the sampling surface may be active on multiple surfaces, for example, on both sides or around in the case of the comb like structure mentioned above. In one or more cases, it can prove advantageous to laminate two sheets together with an impermeable tie layer to increase the binding potential relative to the detachment potential by avoiding flow through the sampling surface. Additionally, further advantages may be provided in other ways such as by increasing the thickness of the sampling material. In some cases, designing samplers in devices such as filter housing may be provided. In other cases, placement on a sampling surface in an active area of the wash system may be provided for getting a full measure of the cross contamination potential.

A sample may be taken after the wash system and will reflect a residual population. In this area there are a number of specific locations that can be considered depending on the specifics of the line. For example, these specific locations include just prior to loading dryers, in a conveyer that might be used to lift the product for packaging, just before a pocket scale, or in the throat of a form fill and seal machine. This in-line continuous sampling may significantly increase sampling efficiency and provide more meaningful data than grab sampling of finished product testing.

With samples taken, attention may turn to the analysis and interpretation of results as discussed herein. For example, in the specific category of produce, there are specific opportunities to be considered that may be provided with the aggregated sampling using the aggregated sampler. The opportunities are afforded in part due to the more representative nature of the aggregated samples and the greatly increase numbers of organisms available in the samples relative to the typical grab sample. These samples may be analyzed to give multiple channels of data depending on the detector technology employed. In one or more cases, the sample may be analyzed with metagenomics, allowing for the whole population to be studied yielding a large and in some cases a maximum amount of data which can be mined in various ways to gain knowledge and understanding of positive and negative deviations. This range of possibilities can be illustrated with a number of examples but are not limited thereto.

In one or more examples, the samples taken in the field, at harvest, or just prior to washing can be used to assess the microbiological status of the raw material. From a food safety perspective the focus heretofore has been on the presence or absence of pathogens. Unfortunately, such analyzes based on grab samples are unable to truly answer the question as to whether pathogens are present due to their lack of sensitivity. Generally, it is known that pathogens are present at very low numbers. These are ubiquitous organisms. A more appropriate question is whether these organisms are present at abnormal concentrations or without the usual competing organism.

In one or more examples, samples taken just prior to washing may be compared to samples taken in the wash system to directly measure cross contamination using wild type flora. Water samples may tend to have very low microbial loads in properly managed wash systems even when cross contamination is occurring. The wild type flora may also be highly variable. However, by using aggregating samplers at both locations the noise can be dampened and cross contamination measured. A number of metrics for cross contamination can be considered based on the ratio of the results for the samples from prewash to those from in the wash. By using more sophisticated analytical procedures one can overcome the flaws in such metrics as aerobic plate count (APC) which would include many organisms that are not relevant to cross contamination control of pathogens. For example, spore forming bacteria such as Bacillus will be unaffected by the wash and just cloud any metric of cross contamination based on APC. However, with more focused channels of data as afforded by modern molecular techniques better information can be obtained. Another aspect of this tool that may be provided is that statistical process control can be applied to detect deviations.

In one or more cases, samples from after a wash process may provide information about microbial populations on the finished product. These samples may provide a much more accurate assessment of the pathogen risk of the product and better detection deviations. These samples can also be used to check for deviations in the normal flora. In some cases, a ratio between the before wash samples and the after wash samples may be used to assess the impact of the wash process. The aggregating samplers may reduce the noise and as with the cross contaminations metrics, these ratios can be handled with statistical process control to look for deviations.

One or more of these examples may be delivered in almost real time because the aggregating samplers collected enough cells for concentration and direct analysis without enrichment.

Examples of Sampling: Bench Scale Examination of Sampling Efficiency for Raw Beef

In accordance with one or more cases, an example of a bench scale study demonstrates a sampling efficiency of 15-20% relative to stomaching. It also shows that transfer is essentially instantaneous and that repeated contact collects more organisms. These observations confirm the intuitive assertion that continuous sampling will yield better information than grab sampling.

For example, for one bench scale study experiment, purchased stew meat is inoculated by immersion in a mixed culture of generic E. coli at ˜10⁵ CFU/ml. E. coli is selected as benign and easy to enumerate. The stew meat is allowed to rest at room temperature for 30 minutes to allow adherence. All sampling cloths may be cut in half, 12 in by 8 in, to reduce the sample requirement and facilitated execution of the experiment. The sampling cloth may be, for example, a MicroTally® cloth but is not limited thereto. The surface areas of the meat cubes are measured directly. For the sampling cloth treatments, meat is arranged in a 10 cm by 10 cm block and the sampling cloth is applied to the upper surface. Each mini sampling cloth is extracted in 200 ml of phosphate buffered saline (PBS). As a control, cubes of stew meat are stomached for 60 seconds in 200 mL of PBS. All stomached cubes are measured to estimate surface area. The counts are normalized for surface area and averaged. This normalization provides an apple to apple comparison.

This bench scale study experiment has been implemented and the average results tabulated. These were analyzed with a General Linear Model (GLM) model which indicates that time of contact was not a factor. Multiple contacts yielded about the expected increase and are truly additive.

A logical extrapolation of this exercise is to estimate the effective sample size of a sampling. This may not truly be possible given the differences in geometry. However, in one or more use cases when using one side only, a sampling cloth is about 6 times larger than those used in the bench scale study experiment and the intended use is to sample almost 2000 pounds of product. Thus, it is reasonable to assert that the effective sample is expected to be 300 to 400 pounds. Larger scale experiments may be implemented that may further confirm this estimate. In summary, a benefit and advantage of the above method and apparatus of this aggregating sampling may include providing an improvement over traditional grab samples for meat sampling by providing larger effective samples.

Examples of Sampling: Bench Scale Examination of Sampling Efficiency for Lettuce

In accordance with one or more cases, an example of a bench scale study demonstrates a sampling efficiency of about 30% relative to stomaching. It also shows that transfer is essentially instantaneous and that repeated contact collects more organisms. These observations confirm the intuitive assertion that continuous sampling will yield better information than grab sampling for lettuce.

For example, in accordance with a bench scale study experiment, purchased lettuce is inoculated by immersion in a mixed culture of generic E. coli at ˜10⁵ CFU/ml. E. coli is selected as benign and easy to enumerate. The lettuce is allowed to rest at 4° C. for 30 minutes to allow adherence. This short time may explain the higher efficiency observed when compared to a meat study where adhesion maybe faster. Treatments may be executed in replicate. All sampling cloths may be cut in half, 12 in by 8 in, to reduce the sample requirement and facilitated execution of the experiment. The sampling cloth may be, for example, a MicroTally® cloth but is not limited thereto. The surface areas of the lettuce leaves may be measured directly. For the sampling cloth treatments, lettuce is arranged in a 10 cm by 10 cm block and the sampling cloth applied to the upper surface. Each mini sampling cloth is extracted in 200 ml of PBS. As a control, lettuce may be stomached for 60 seconds in 200 mL of PBS. All stomached leaves are measured to estimate surface area. The counts are normalized for surface area and averaged. This normalization provides an apple to apple comparison.

Bench scale study experiment were implemented and average results are tabulated and analyzed with a GLM model which indicates that time of contact was not a factor. Multiple contacts yielded about the expected increase and are truly additive.

A logical extrapolation of this exercise is to estimate the effective sample size of a sampling. This may not truly be possible given the differences in geometry. However, in one or more use cases when using one side only, a sampling cloth is about 6 times larger than those used in the bench scale study experiment and the intended use is to sample almost 2000 pounds of product. Thus, it is reasonable to assert that the effective sample is expected to be 400 to 600 pounds. Larger scale experiments may be implemented that may further confirm this estimate. In summary, a benefit and advantage of the above method and apparatus of sampling may include providing an improvement over traditional grab samples for leafy sampling by providing larger effective samples.

In one or more cases, swabs and/or sheets, referred to generally as a sheet below, may be used in accordance with one or more embodiments of the present disclosure. A sheet may include a microbial sampling material, such as sterile woven and/or non-woven synthetic fabrics and non-woven cloth for sampling and testing in the field of food safety. These sheets can be folded and curved to allow better conformation to product (e.g., food) streams that are being sampled or when used in a manual mode as driven by the product and the container. In some cases, configurations for sampling raw products or materials may include a sampling device that moves across the stationary product effectively yielding the equivalent of a product stream when sampling needs to occur prior to harvest. In some cases, configurations may include tail shaped sheets similar to, for example, the tentacles of a jellyfish.

In one or more cases, configurations may be provided that may provide easier use under some conditions. In one case, a microbial aggregating sampler, may include a covering including a microbial sampling material with a pocket formed in the covering to receive an appendage or a tool for handling of the covering. In some cases, the covering may further include an attachment feature formed in the pocket to receive the tool. The attachment feature may include at least one of a hole formed through the covering; a loop positioned within the pocket to receive an end of the tool there through; or a tab positioned within the pocket for an end of the tool to attach thereto. In some cases, the covering may include a sheath formed in the pocket to receive a digit of an appendage. In some cases, the pocket is formed through the covering such that the appendage or the tool for handling the covering extends through the covering. The covering may be completely formed from the microbial sampling material. In some cases, the covering includes two sheets attached to each other to form the pocket. In some cases, the covering may include a single sheet folded and attached to itself to form the pocket.

For example, a sheet may undergo folding and seaming to form a bag or pocket that can be worn as a mitten, glove, sock, or other covering, to facilitate manual sampling. Such a covering may encase an appendage, such as a hand, to facilitate pushing and pulling of the sampler through the product to be sampled. In some cases, aggregating samplers may be created that are more hand like configurations as mitts with thumbs and/or gloves with fingers, allowing the sampler to better conform to the hand. Such configurations may allow easier use when the product is more viscous or more prone to adhering to the sampler. A benefit to this aggregating sampler with a pocket may include the ability of the sampler to, when working the sampler, increase or maximize product contact. Further, in some cases, the addition of one or more attachment features, such loops or tabs, to assist with controlling the product contact may be included. These are representative examples and are not meant to limit other configuration embodiments of an aggregating sampler.

In some cases, the aggregating sampler may be used in an automated machine setting to sample a product stream. The aggregating sampler may include a number of modifications in accordance with one or more cases. In some cases, the aggregating sampler may include one or more bends and curves that may improve the utility and facilitate use. In some cases, modifications may include, but are not limited to, forming a tube that can slide over one or more shafts for positioning in the product flow. This configuration may remove the need to slide the sampler within any holding device. In some cases, adding tabs or holes can be provided which may allow positioning and facilitate attachment. In some cases, the active sampling surface can be attached to a support material or web that has one or more of these features. One or more of these cases and modifications may help facility contact with the product stream with minimal manual intervention.

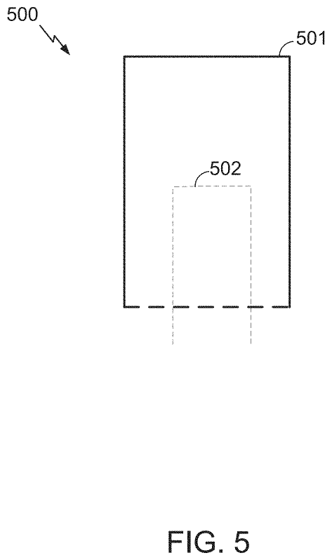

FIG. 5 illustrates an aggregated sampler 500 that includes a pocket in accordance with aspects of the present disclosure. As shown the aggregated sampler 500 includes a cover 501 made of a microbial sampling material. The covering is formed such that it includes a pocket formed in the covering to receive an appendage or tool 502 within the pocket for handling of the covering.

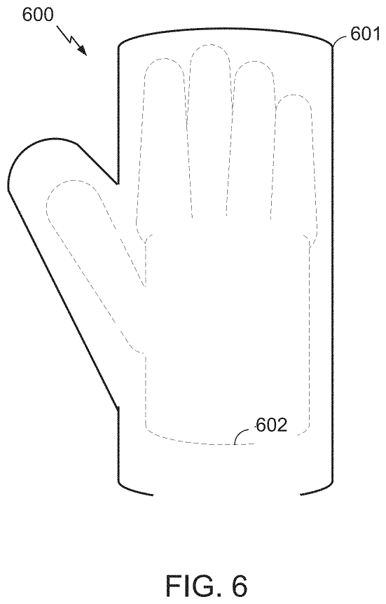

FIG. 6 illustrates an aggregated sampler 600 that is shaped as a mitten or glove for a hand 602 in accordance with aspects of the present disclosure. As shown, the covering 601 of the aggregated sampler 600 is formed to contour to a hand 602. In particular, a sheath is formed in the covering 601 in this embodiment to receive a thumb of the hand 602, though one or more sheaths may be used to receive any digit of the hand. Further, though only a hand is shown as an example appendage in this embodiment, the present disclosure is not so limited, as other appendages (i.e., a foot) may be contemplated for other embodiments without departing from the scope of the present disclosure.

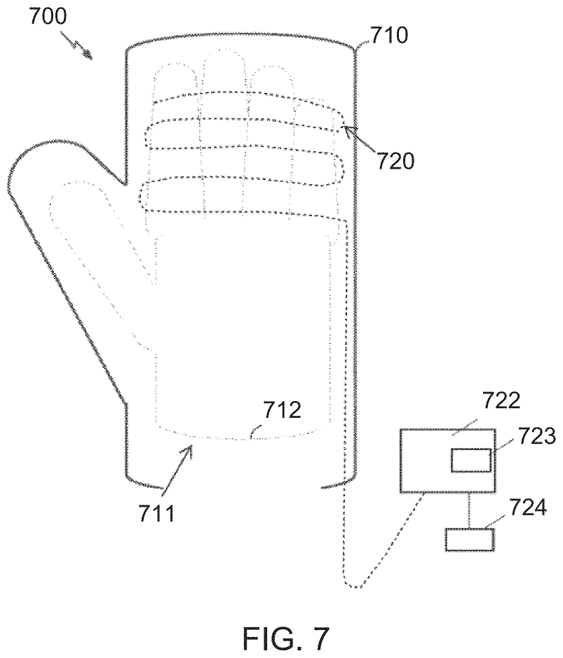

FIG. 7 depicts a heating sampling probe device 700 shaped as a glove 711 that includes an interior pocket 712 contoured for receiving a hand of the sampling personnel. The glove includes a resistive heating element 720. An outer sampling cover 710 fits over the glove and heating element 720. The cover is fabricated from an absorptive sampling medium suitable for sampling a food product. In this embodiment, the cover resembles a mitten and can be slipped over the glove. The cover can include an interior liner that is impervious to fluid to protect the heating element and internal glove from any liquids. The resistive heating element 712 is a flexible element that extends across the fingers of the glove and is electrically coupled to control unit 722 having a thermostatic controller 723 to maintain a suitable elevated temperature and is powered by a power source 724. The power source 724 can be an external power grid or a portable battery, as descried herein. This sampling probe device can be used for obtaining sample from frozen food products, without requiring full thawing of the entire product being sampled.

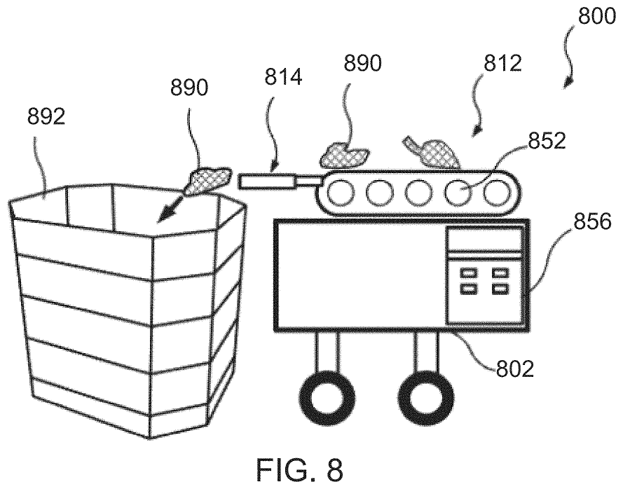

FIG. 8 shows another type of aggregating sampler, which can be used as a continuous sampler that remains stationary relative food products being conveyed. The sampler can also be disposed on mobile cart so as to be movable to various locations along a food transport path. Accordingly, with reference to FIG. 8, aggregating sampler 800 can be used with a conveyor to provide consumable products 890 to a bin 892. The aggregating sampler 800 includes a mobile frame 2502 with a guide surface 812 and a sample surface 814. The guide surface 812 can include a guiding conveyor 852 that moves with respect to the mobile frame 802 to convey the consumable products 890 from the guide surface 812 to the sample surface 814. The sample surface 814 can include one or more sampling sheets that can be periodically replaced between each bath of food products being conveyed.

It is noted that the above are exemplary sampling approaches to collecting an aggregate sample, and that various other approaches and devices could be realized. It is further appreciated that the inventive concepts described herein are not limited to aggregate samples and can also be used to improve testing of various other types of samples, including discrete, non-aggregate samples, such as grab samples or spot sampling.

In accordance with one or more cases, the aggregated sampling sheet may be hung into an active zone of a wash line with a cord or cable. This line may be attached in number of ways to the sampling material such as, for example, a grommet and clasp. In some cases, a ball and slide clamp may be used. In some cases, a punched hole may be used but may pull out. In other cases, various other clamps may be used. In one or more case, an optional float such as a fishing float with or without a weight may be included that will add drag and bouncy which may improve surface exposure. This approach may not include a fixed appliance for holding the sampling material.

Example of the Special Case of Sampling Frozen Materials

To sample frozen materials, it is not necessary to completely thaw the material in cases where the surface layer is representative of the whole or where the surface layer is the expected portion of the material expected to contain the target analyte. In general, these are the conditions that prevail when aggregated sampling is effective. Aggregated sampling of a frozen material can be achieved by melting the surface layer and relying on the thermal resistance of the product to maintain the frozen condition of the bulk of the material. The melted surface layer is placed in intimate contact with the aggregated sampling media transferring a portion of the analyte to the sampling media. To aid in using this method, it is appropriate to consider the two elements and the logistics of use.

The energy source for melting is the heart of this sampling method. Thermal energy is the most direct but conceptually other radiant energy such as microwave or light can be used. The complexities of using the other forms of energy may be offset by reduced contact with the product. One might consider the samplers body heat from their hand, but this rapidly becomes inadequate particularly when multiple samples are collected in a short period of time. Concepts that are compatible with the need include heated or recirculated water, electrical heating, chemical heating, hot air, near/far infrared, or fuel combustion. The use of each of these energy sources is elaborated below.

With of all these energy sources it is important to balance the delivery of energy with the need for temperature control. Too much temperature can damage the product and or the analyte. With too little energy, the desired melting of the surface layer for sampling will not be achieved. It may be useful to consider these two constraints as the quality of the energy. Typically, a frozen product refers to a product with water ice. However, any solid material with a suitable melting point could be sampled in this way if it can be absorbed on the sampling media and if the absorbed analyte can be analyzed. Focusing on the primary case where water ice is the solid to me melted, delivered temperatures less than 45 degrees C. are amenable to most analytes including most bacteria. Water ice nominally melts at 0 degrees providing a temperature differential window to drive melting. Clearly these temperatures are only indicative, and higher temperatures may be tolerated in some cases with more robust analytes such as when chemical constituents are to be analyzed. The melting point of water ice can be depressed with solutes as freezing point is a colligative property. It should be noted that with higher temperatures the rate of melting will increase allowing faster movement of the sampling media over the surface of the solid or frozen material.

Circulating water or other liquid with a suitable specific heat can be used to transfer energy to the surface of the material to be sampled. Water is generally preferred due to its high specific heat, availability, and low toxicity. The temperature of the liquid can be controlled in a temperature-controlled bath as desired. Clearly other liquids can be used such as oils if there are specific requirements for sampling. One such restriction would be to sample a material with a melting point over 100 degree C., the boiling point of water. The heated liquid can be pumped through a bladder or other heat exchanging design covered by the sampling media discussed below at a rate sufficient to melt the surface of the solid sufficiently to sample the surface. This device is normally flexible enough to conform to the potentially irregular surface of the material to be sampled but firmer sampling devices can be desirable. Increasing the flow of the liquid will deliver more energy and therefore greater melting. The bladder can be a simple bag or can have a more restricted path to provide more uniform temperature depending on the need. The configuration needs to be tailored to fit the configuration of the sampling media. The portability of this system may be limited due to electrical power requirements and the mass of the liquid reservoir. However, cart mounting, and battery power may be sufficient to allow sufficient portability.

Direct electrical heating can be achieved by embedding electrical heating elements in heat transfer blocks or in a user friendly mitten. Temperature control can be achieved with thermocouples, thermal resistors or mechanical thermostats systems. The range of systems for such temperature control are well known and need not be elaborated here. Here again the heating blocks can at need be articulated to allow flexibility to conform to irregular surfaces and to fit the configuration of the sampling media. Here again a user friendly mitten will reduce hand strain. This use of electricity may best be handled by using power from the electrical grid, but some degree of portability maybe achieved with batteries.

Chemical heating such as used in the hand warming packets that are in commerce can be used as a heating source. These packets generally rely on the heat of dissolution of a salt to provide heat. Similar technology is used for heating food where other sources of energy are inconvenient. These packets are generally activated by rupturing a container within a container to initiate the chemical reaction. A packet of this type can be placed in the sampling sleeve to achieve melting to sample the surface of the material. These packets could be single use for one sample or transferred to other sampling media sleeves for additional samples depending on the energy content. The compositions of these packets are available elsewhere and will not be elaborated here. This source of energy can be preferred where complete portability for sampling is desired.

The use of infrared is as non-contact, no cross contamination, and as repeat usages to deliver a consistent energy to melt a frozen sample surface for efficient sampling. The energy usage can be optimized, near or far infrared, to melt the frozen sample surface in a short time to allow a use of sampling sleeve or mitten to complete an efficient and sufficient microbiological sample. Care is required to avoid generating uv light would can kill bacteria and therefore invalidate the sampling.

The use of propane or liquid fuel can also be considered but these are less preferred. Combustion or catalytic conversion to combustion products will yield energy but generally it is at higher temperatures and require quality reduction. However, it should be noted that these reduction in quality have been accomplished so the use of these energy sources can be considered if other conditions warrant their use. Hand warmers based on these energy sources have long been used but have mostly been displaced by chemical packets for safety and convenience.

Heat gun can use generate hot air which applied to the surface will melt the surface layer for sampling. Presumably the heating process will assure that the air does not contaminate the surface but HEPA filtering of the feed air can provide more assurance. The airflow and temperature will allow temperature control.

Sampling media used to fabricate the sampling sleeve needs to transfer the melted surface material without contaminating the energy source unless the energy source is single use as might be the case for the chemical packets. The media will generally be a food contact material for most applications, but other materials may be appropriate for non-food applications. For food application polyolefin materials such as non-woven polypropylene fabric would be appropriate. Sonic welding can form a wide range of sleeves for covering the heating element. The shapes can be as simple as an open bag to something with appendages more like a glove. The configuration will be defined by the surface irregularities of the material to be sampled. Cellulose materials can also be used to fabricate sleeves that are appropriate for food contact.

Generally, to prevent contamination of the heating element, the sleeve will be lined with a plastic web. This material can also be approved for food contact, but other materials can be considered. Again, the polyolefins are the preferred choices being easier to work. Again, for non-food applications many more plastics webs become acceptable. This inner lining can be attached to the sampling sleeve media or just inserted into depending on the fabrication process.

As a validation step for aggregated sampling, a before and after weight of the sampling sleeve or media can determine the weight of material during the sampling process. Care must be taken to avoid other changes in the weight such as those due to a tear off strip for the bag. In many cases, the automation of the manufacturing process will provide a consistent before weight and an after-sampling weight is sufficient to ensure that the sampling was properly executed. This is of particular importance when the analyte is a negative attribute such as the presence or absence of pathogens. Too little weight gain would indicate too little material was transferred.

In sampling, it is critical to avoid contamination. Contamination is always related to the planned testing or the analytes. Generally, one avoids contact with surfaces other than the material to be sampled. For microbial testing, this means that aseptic technique is used. It also means that the sampling sleeve must be hygienic relative to the organisms to be tested. As a practical matter, this usually implies that the sampling sleeve is sterile. Sterilization can be achieved in many ways including chemical such as ethylene oxide, radiation such electron beam, or heat. Other techniques may be appropriate in particular instances.

The operator or user will almost certainly be wearing gloves while using the sampling device. Protecting the material and the sampling sleeve from contact with the operator is part of preventing contamination. In some instances, other protective equipment will be appropriate.

Given that this sampling technique depends on various differential temperatures, it can be beneficial to temper the product to reduce the specific heat requirement to achieve surface melting. Expressed differently, the sampling is easier if the surface of the material to be sampled is closer to the melting point than if the material is much colder. Thus, in the beef trim example described below, the frozen trims have been removed from the −20 deg C. storage for several hours to let the surface temperature rise while the bulk of the product remains much colder.

There are many instances where this sampling approach can be useful. The application to frozen beef trims is examined more fully below. As another illustrative embodiment of the invention, the authentication of an apple juice concentrate can be considered. Apple juice can be adulterated with a variety of sugar syrups that can only be detected by specialized procedures. High quality apple juice is generally stored under frozen conditions. It would be desirable to confirm the authenticity of the product without the need to thaw several boxes of the product.

The sampling procedures are similar. The processing need not be aseptic but still contamination needs to be avoided. Heated sleeves are used to collect the surface melt of the concentrate. This melt is extracted into water and dried under nitrogen. The residue mostly sugars and other carbohydrates is derivatized to gas-chromatographic analysis of the complex carbohydrates looking for the finger prints of materials such as hydrolyzed inulin syrups. The ratio of juice constituents will also need to fall within the norms. In this way, apple juice concentrate can be authenticated without a full thaw.

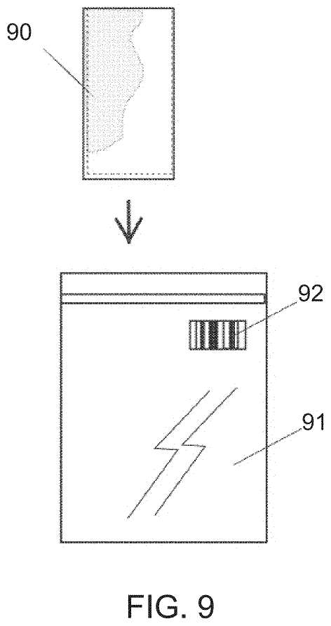

In another aspect, regardless of the sampling approach or type of sample collected, the sampling medium is collected, and as shown in FIG. 9, the used sampling medium 90 is placed in a sample bag 91 having a sample identifier 92 (e.g. barcode) on the bag. The sample identifier can be entered or received into an automated or computerized sample tracking system. The sampled medium can be processed by suitable techniques (e.g. washed, extracted, etc) to obtain the fluid sample to be filtered, concentrated and tested. In some embodiments, the fluid sample can be obtained after enrichment, for example in a nutrient rich both disposed in the bag. Preferably, the sample can be tested by a procedure that extracts and concentrates the liquid sample for direct testing of the analyte of interest without requiring any enrichment procedure. Since the sample processing techniques described herein allows for improved recovery of target molecules from the fluid sample, enrichment is not required, which greatly improves efficiency and speed of sample processing and testing. This is particularly advantageous in food processing, where food products must be processed and transported to consumers as quickly and efficiently as possible.

Examples of Sampling: Environmental Monitoring Programs

In another aspects, sampling and microbial analysis are important aspects of an Environmental Monitoring Program (EMP). Most such sampling is done with handle mounted sponges urethane or cellulose sponges (e.g. 3M Sample Collection: Microbial Surface Sampling, Environmental Testing 3M). Recently other offerings are being made such as the 3M scrub sampler where a sponge is enclosed in a synthetic fabric. In some embodiments, such programs can utilize a specialized sample vial coupled with a powered sampler (e.g. FREMONTA vial associated with Smart Sampler 300 Series). The Smart Sampler vial uses a non-woven polyolefin fabric that provides better collection and release of organisms for better sampling. This same material can be mounted on a handle and provide better results than the normal urethane and cellulose materials. This non-woven fabric has the further advantage that it is fully approved for food contact.

EMP samples are traditionally enriched for 12-24 hours to allow any pathogens present to increase to readily detectable numbers. These delays can hinder many EMP activities particularly investigations of contamination. Listeria and Salmonella are the most common organisms of interest. These are readily detected by various molecular techniques with PCR being the most common.

Advantageously, EMP samples can be readily treated and analyzed by the methods described herein. These samples are essentially free of tissue. Extraction procedures yields a good sampling of the collected organisms which can be readily concentrated in a manner described herein (e.g. similar as to MicroTally® swabs or MicroTally® mitts). Thus, the sample treatment and testing methods described herein are further compatible with EMP samples, thereby providing sample treatment without requiring enrichment within two hours or less, allowing for rapid on-site testing.

Examples of Extraction

The reference method for extracting microorganisms from grab samples is homogenization by mixing or stomaching with an appropriate amount of fluid, usually a buffer. The purpose of adding fluid is to neutralize any antimicrobial or other properties of the sample that may be unfavorable for microbial growth such as low pH and suspend microorganism in liquid to facilitate downstream testing. However, adding fluid also dilutes the concentration of the microorganisms in the sample. Because most microbiological testing only take a portion of the homogenate (e.g., 0.1 mL or 1 mL for plating; 2.5 μL for direct PCR), the homogenization by dilution method decreases the detectability of the organisms of interest by as much as 1,500,000 times. To increase the detectability, a lengthy enrichment procedure is incorporated to allow a single viable cell to proliferate to millions so that it can be detected. This procedure costs 24 to 48 hours delay in obtaining detection data and more days in decision making time due to subsequent confirmation steps. It is also important that the samples be preserved properly and extraction be done as close to sampling time as possible. If the sample has changed before extraction, the results do not represent the tested lot.

For most conventional sampling programs, the percentage of negative samples exceeds 99%. Based on simple modeling, one can easily conclude that the number of cells of the organism of interest is seldom more than 1 and probably rarely more than 5 based on a Poisson distribution. With so few cells present, the enrichment is often done in the extraction buffer. This is critical with the small sampling but does not make this type of testing any more representative of the lot under test.

However, this conventional enrichment and detection can be applied to extract from an aggregating sampler when presence or absence of the target organism is the desired goal. Modes of detection are considered herein.

When extracting the sampling materials these same considerations apply. However, the impact on the results of failing to extract one organism is an order of magnitude or smaller due to the larger effective sample. In addition, the cassettes afford the opportunity to begin the extraction faster by adding the fluid immediately after sampling providing maximal time for the extraction to occur while avoiding human interaction.

When extracting a 24×8 inch sampling sheet of a poly-olefin sampler, a volume of 100 to 200 mls of extraction solution is generally appropriate. The composition of this solution is driven by the planned detection system and is generally defined in the associated method.

Examples of Concentration Methods

During the concentration, two classes of materials may need to be removed, the small, <100,000 molecular weight, and the very large, >50 microns. In addition, the sample may need to be concentrated to about 1 mL to be compatible with the screening detection system. Given that the extraction generally starts at about 200 mL, there is a large amount of water to remove.

Two schemes are practical. Traditionally, one can filter the extraction fluid through an inert 50 microns cut off filter and then sediment the organisms of interest by centrifugation. The resulting pellets can be re-suspended in an appropriate buffer and taken on to cleaning. Alternatively, after filtration, the small molecules and water can be removed osmotically with adsorbents or pressure and a semi-permeable membrane such as used for reverse osmosis or ultrafiltration. The latter is more amenable to automation as the resultant concentrated sample remains in solution. However, this option may require a for purpose module to be executed.

It is important to maintain the connection to the initial Meta data through this process. If enough of interfering material is removed and the sample is sufficiently concentrated, the cleaning step examined next can be skipped and moving directly to the screening determination. This decision determination may be made of a case by case basis.

It is also possible to use non-specific binding such as a cation exchange surface to collect that target organisms and remove them from the bulk solution. Such as an approach would partially combine concentration and cleaning. This approach is most practical when the there are few larger particles to interfere.

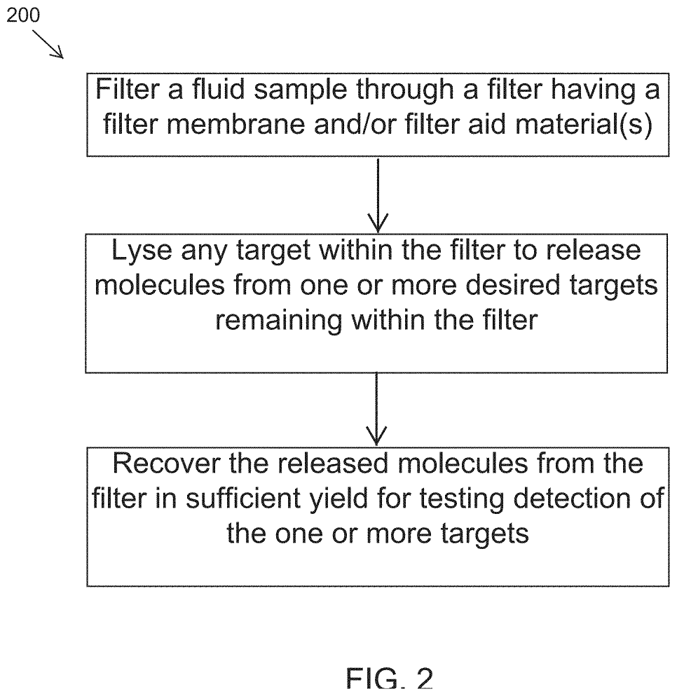

FIG. 2 depicts a method of concentration utilizing filter aid materials in accordance with the workflow depicted in FIG. 1. The general method includes steps of: filtering a fluid sample through a filter having filter aid material(s); lysing the target material trapped within the one or more filter aid materials and/or filter to release molecules from one or more desired targets remaining within the filter aid material(s); and recovering the released molecules from the one or more filter aid materials in sufficient yield for testing detection of the one or more targets.

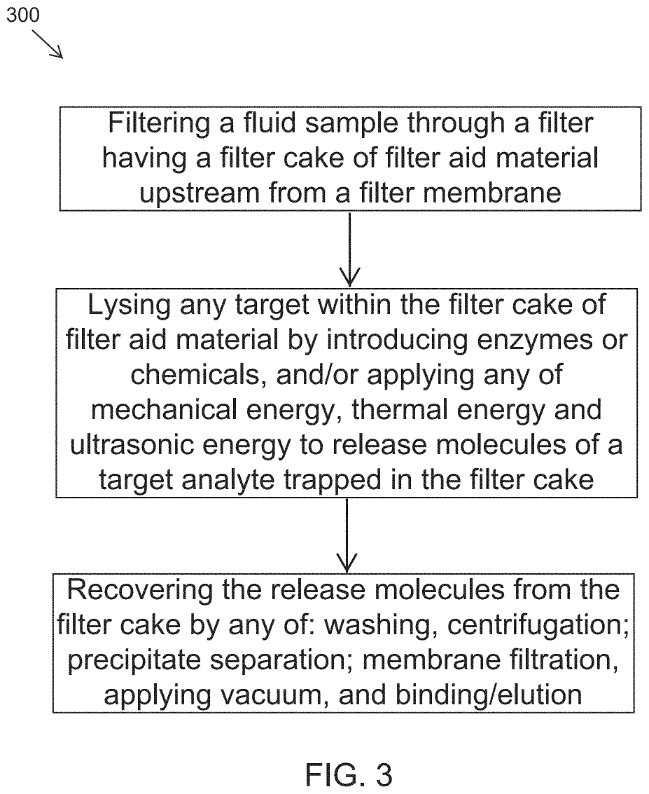

FIG. 3 depicts method 300 which includes additional details regarding concentration utilizing filter aid materials. Method 300 first, filters the fluid sample through a filter having a filter cake of filter aid material upstream from a filter membrane. The filter aid cake can be formed of one or more filter aid materials, including but not limited to: perlite, cellulose, diatomaceous earth, vermiculite or other porous materials. Next, the filter cake is lysed to release molecules of a target analyte trapped in the filter cake. Lysing can include introducing enzymes or chemicals, and/or applying any of mechanical energy, thermal energy and ultrasonic energy. Next, the released molecules are recovered from the filter cake by any of: washing, centrifugation; precipitate separation; membrane filtration, applying vacuum, and binding/elution.

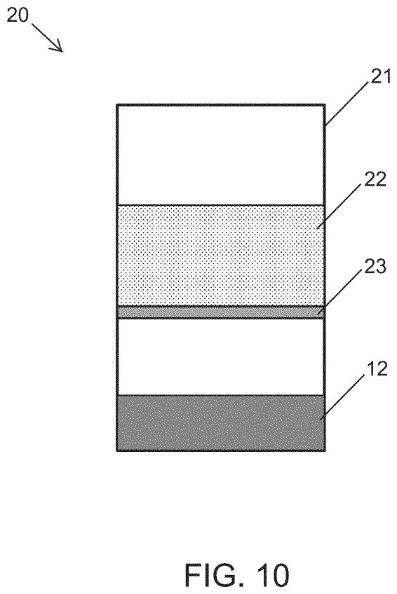

Additional aspects of improved methods of concentration utilizing processing of filter aid materials are described throughout, and are depicted in FIGS. 1 and 10. FIG. 10 depicts an exemplary filter device for concentrating the fluid sample utilizing filter aid materials. As shown, filter 20 includes a container or conduit 21 that includes filter aid material(s) 22 (e.g. filter cake) disposed upstream from a filter membrane 23 so that the fluid is filtered through the filter aid material(s) before passing through the membrane and being collected as concentrated fluid sample 12. In some embodiments, the filter aid materials(s) can be removed from the filter 21 and processed (e.g. trapped target material is lysed) to recover released molecules from the target. In other embodiments, the lysing means can be applied to the filter aid material(s) in situ, so that the lysing and recovery processing can occur within the same filter 21. This concentration can also be partly combined with cleaning processing, as described further below.

Aggregating Sampler for Vertical Filling/Sealing System

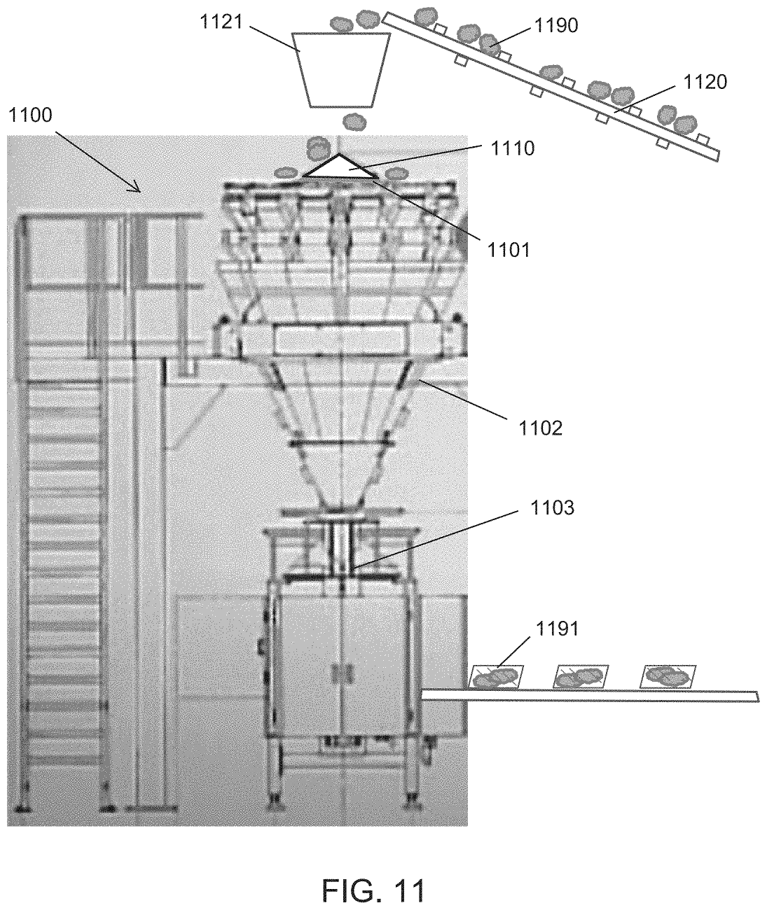

In yet another aspect, the sample can be obtained from an aggregating sampler configured for a vertical filling and sealing system used for bagging fresh cut produce. Fresh cut produce presents some special opportunities for aggregated sampling. The low incidence of pathogens and the demands of the marketplace to test finished product provide an opportunity for improvement. Large portions of the industry use vertical form fill and seal machines, such as that shown in FIG. 11. In such systems, the produce is fed to the fillers from a vibrating screen which drops the product on a cone or “hat”. Advantageously, a cone shaped sleeve sampling member can be secured on this hat to effect aggregated sampling.

FIG. 11 shows an exemplary vertical form filling and sealing system 1100 for bagging fresh cut produce. The system 1100 includes a conveyance means 1120 for transporting the fresh cut produce through a chute 1121, which then drops onto the upward facing cone or hat 1101, which directs the produce into a downward facing cone 1102 that sorts and directs the produce to fill individual sealable bags, which are then heat sealed in sealing portion 1103, to produce a individually sealed bags 1191 of fresh cut produce. Such produce typically includes, lettuce, leafy vegetables, but can include any type of produce or products processed in this manner. A sampling member 1110 is disposed on the hat so that the produce dropped from the chute 1121 contacts the sampling member 1110 to obtain an aggregate sample. After processing of an entire batch, the sampling member 1110 can be removed and tested and the result can be associated with the processed and bagged batch of produce. The sampling member is typically an absorbent or porous material or fabric, such as a non-woven fabric, various micro fiber materials, sponges, and/or any absorbent sheet material. A non-woven polypropylene or polyethylene fabric are of particular note as these materials are allowed for food contact and therefore have very low extractables which might otherwise contaminant the material stream under examination.

To conform to the shape of the hat in the vertical filling and sealing system, the aggregate sampler can be defined as a conical sleeve. This cone shaped sleeve is fabricated by removing a sector from a circular disk of sampling material but leaving a small tab. This tab can be attached (e.g. by sonic welding or adhesive) to the opposite side of the removed sector to form a cone with the desired angle for fitting the hat. The tab can be separately formed or can be an integral portion of the disk. In some embodiments, the radius of the initial disk is selected to correspond to the length of the side of the cone. In some embodiments, the radius is selected so that the sampling member covers at least 50% of the cone, at least 75% of the cone or substantially the entire upwards facing surface of the cone that contacts the produce. In some embodiments, the radius of the disk is between 6 inches and 3 feet, typically between 1-2 feet. In some embodiments, the removed section matches the remaining circumference of the disk to the circumference of the cone. While a specially shaped sampling member is described herein, it is appreciated that in other embodiments, the sampling member can be a standard sampling sheet (e.g. rectangular sheet, such as a 24×8 in sheet) of any suitable material (e.g. non-woven cloth, polypropylene fabric), such as a MicroTally® swab, that is secured to the hat, or an upward projecting knob at the apex of the hat, by any suitable coupling or retention means (e.g. fastener, retainer ring, screw-on cap, slit, frame, etc.). In some embodiments, the sampling member includes a slit or hole that fits over an apex or knob of the China-cap or hat of the vertical filling and sealing machine, thus this approach can be referred to as China-cap sampling

China-cap sampling is a special form of continuous sampling device (“CSD”) testing where a sample medium cloth (e.g. Micro-Tally® swab) has an opening (e.g. one or more slits) to allow placement over the China-cap or hat of a multi-pocket weigh scale. The weigh scale has a central cone, the China-cap that distributes product to the computer-controlled pockets of the weigh scale when product is conveyed to the weigh scale. The product, typically produce, is dropped onto the China-cap where is flows out radially. Such vertical fillers can be used for a wide variety of products including leafy greens, chopped or cut onions, various dry products such as rice, nuts, or dried fruits, and many other products both food and non-food items that are filled into packages of various type that pass under the chute of the weigh-scale.

When it is desirable to collect an aggregated sample of the surface of the product passing into the weigh scale, a sample member (e.g. MicroTally swab) can be effectively attached to the China-cap by cutting one or more slits (e.g., a slit, two slits or three slits or more) at or near the middle of the sampling member. These slits need to generate enough space to slide over the top of the cone or over a knob that is optionally over the apex of the China-cap. It is not required that the swab cover the entire cone just enough of the cone to generate a representative sample of the surface of the product. The downward force of the product holds the swab in place. If a single slit is used, the slit should be of sufficient dimension (e.g. 1″-12″, typically 3″-6″) to allow the swab to slide down suitably onto the cone. In some embodiments, two or three slits are used as it allows the top of the cone or knob to more easily penetrate the swab with less deformation. Although most any angle could be used, two orthogonal slits would advantageous over other angles as it distributes the forces in two orthogonal directions. For three slits, again any angle could be used but three slits offset by about 60 degree are advantageous as this distributes the forces more evenly. Although more slits can be used, the addition of additional slits adds little to the utility of the swab for China-cap sampling. In recent studies, slit lengths of less than 5 inches were used, however, it is appreciated that the length of the slits is a function of the actual sampling opportunity (e.g. larger China-caps can utilize swabs with longer slits and vice versa).

In another aspect, the duration of China-cap sampling is largely a function of lot size. Care should be taken that the swab is not saturated or that the target is not diluted too much by overly long durations of sampling. Swabs have been used for very short sampling periods and for up to several hours to great advantage.

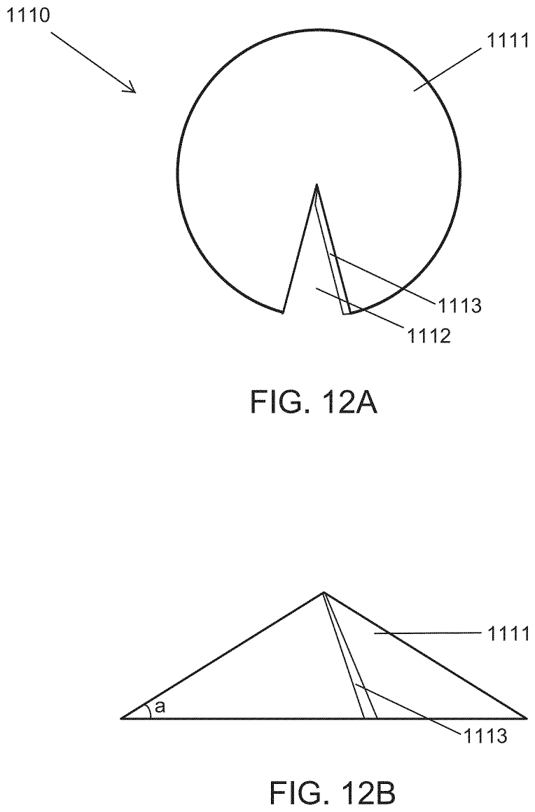

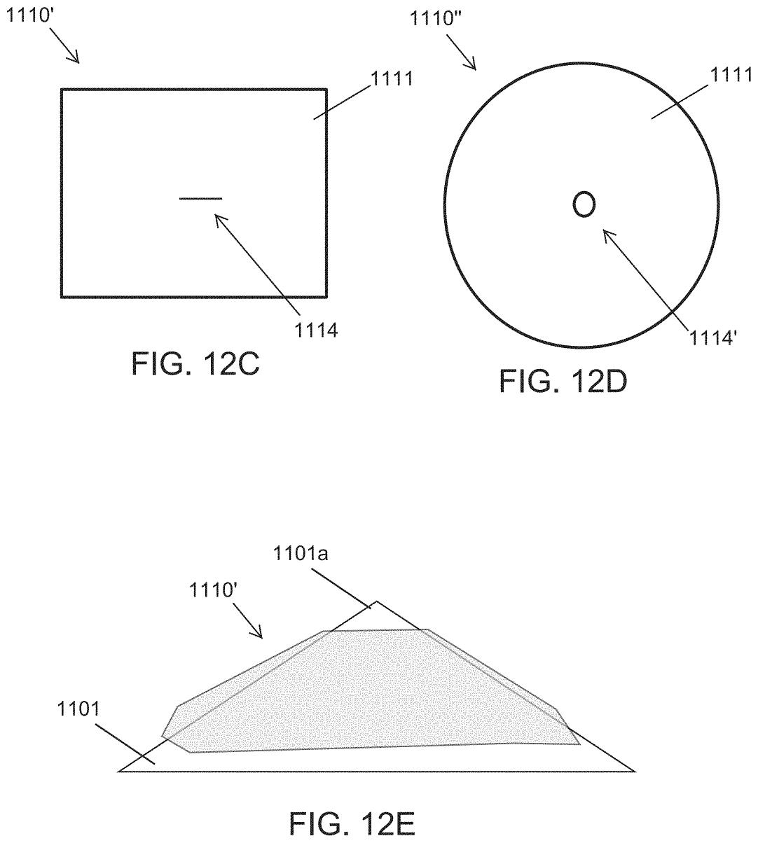

FIGS. 12A-12B show an exemplary sampling member 1110 that is shaped as a conical sleeve for placement atop a conical “hat” of a vertical filing and sealing system for produce in order to facilitate aggregating sampling. As can be seen in FIG. 12A, the sampling member can be configured as a circular disk 1111 of porous or absorbent sampling material with a removed sector 1112 and a tab 1113. The size of the sector removed is selected so that when the tab 1113 is joined to the opposite side of the removed sector, the angle a corresponds to the incline angle of the hat. In some embodiments, the angle a is between 20 and 80 degrees, typically between 30 and 60 degrees, typically about 45 degrees. It is appreciated that the sampling member 1110 need not be circular in shape and need not be configured to precisely match the shape of the conical hat so long as the sampling member 1110 can be releasably secured atop the conical hat of the vertical filling/sealing system. For example, as shown in FIG. 12C, the sampling member 1110′ can be a rectangular sheet (e.g. a 12″×8″ MicroTally cloth) with a central opening 1114 for receiving an apex 1101a of the conical hat 1101 of the vertical filling/sealing machine, as shown in FIG. 12E. In this embodiment, the opening is formed by a single slit, which is sufficient to receive the apex to maintain the sampling member in place. It is appreciated that the sampling member could be of any shape, such as a rectangle, square, any polygonal shape, or the circular shaped sampling member 1110″ in FIG. 12D, and that the central opening could be defined as by multiple intersecting slits or an opening of any shape, such as the circular hole 1114′ shown in FIG. 12D. Notably, since the produce falls down from above the conical hat, the force from the produce does not dislodge the sampling member from the hat. This approach of releasably securing the sampling member by an integral feature, such as a central opening, is advantageous as it can be easily placed and removed by a user between processing batches of produce and does not introduce additional components requiring sterilization and replacement. This general approach can be referred to as “conical cap sampling” or “China cap sampling” as the sampling member sits atop the conical head/hat.

To avoid the sampling cone or sheet becoming a foreign object in the product or for jamming the weigh buckets of the scale include in the filler, it may be desirable to further provide an attachment means to hold the sleeve in place within the system. It is important to avoid the addition of foreign objects from the clipping mechanism to enter the product stream. This attachment means can include one or more clips, fasteners, interfaces, magnets, any combination thereof, or any suitable attachment mechanism. One simple version is strips of spring steel attached to the cone which can be lifted to allow the sleeve passage to be restrained when the spring steel strip is released. Bending the tip up allows easier manipulation. It is appreciated that many other clipping mechanisms could be employed.

FIG. 13A depicts one approach that includes an attachment means of a ring interface 115 that fits over the lower edge of the conical sleeve sampling member 1110 so as to secure the sampling member on the hat 1101. FIG. 13B depicts another approach of spring-loaded fasteners or clips 1116 that can be lifted to position or replace the conical sleeve 1110 on the hat, each fastener or clip engaging a lower edge of the sampling member. In other embodiments, the tip or intermediate portion of the sampling member can be engaged by the attachment means. In some embodiments, the sampling member is secured by virtue of its shape and engagement with the hat.

These cone sleeve sampling members can be replaced at any suitable interval based on the users desired lot size. More frequent replacement decreased lot size. Interval times between 15 minutes and 2 hours are generally useful in most applications. It is appreciated that these conical sleeve sampling members can be used in accordance with any of the filtering, sample processing and testing methods described herein.

Pilot Study

A pilot study was performed to demonstrate the effectiveness of the above-described sampling approach with a standard sampling sheet, in particular, a MicroTally® swab, to demonstrate improved effectiveness as compared to conventional sampling methods. The results indicate that aggregated sampling with the MicroTally® swab is more effective than the regular tissue sampling at detecting generic E. coli (p<0.01). This increased effectiveness is attributed to the surface sampling of a much larger portion of the product as it passes over the cone of the scale just prior to filling. It is posited that this efficacy would also apply to sampling for pathogen detection.

Generic E. coli was selected as the target organism for this study to allow multiple detections within 200 samples given that pathogens are extremely rare. In practice, the 90 samples collected proved sufficient to confirm the benefits of aggregated sampling allowing early termination of the sampling. Generic E. coli has many similar attributes to the pathogens of interest making it a reasonable surrogate for this study. Samples were not enriched for either the aggregated sampling or the tissue grabs to utilize the low concentration of the target to enhance the separation of the two sampling methods. The concept was to collect a similar number of total organisms by the two methods based on previous experience, which allowed for a direct comparison.

For the aggregated sampling, a MicroTally® swab was placed over the conical hat of a scale/sorting system, such as that illustrated in FIG. 11. This placement allows a significant portion of the product to contact the sampling medium sheet just prior to entering the pockets of the scale/sorting system. Swabs were in place during the period represented by the grab sample, often two to three hours but some shorter times were included. The sullied swabs were shipped overnight to the testing laboratory where the sample were extracted into 150 mls of buffer with stomaching and plated on rapid petri film for detection and enumeration. One milliliter of the 150 mls was tested yielding a measure of organisms per swab with a detection limit of 150 cfu per swab. Two samples were lost during this process reducing the number of aggregated samples to 88. The results were assessed and comparative data for the tissue samples generated. The tissue samples were collected and analyzed as usual for PCR testing. The tissue samples were diluted 1:10 with media. Prior to enrichment, a small aliquot was collected for immediate plating on rapid petrifilm for detection and enumeration of generic E. coli. This procedure yielded a limit of detection of 10 cfu/gm of tissue. The results were largely negative as anticipated. There were two detections for the tissue samples. These positives were not associated with any of the six detections by aggregated sampling. Overall, analysis of the results leads to the most applicable statistical test of difference. For a Poisson variable, the standard deviation is the square root of N. This allows performance of this test with 90 observations which indicates that this difference is highly significant.

Examples of Cleaning

At this point in the process, samples have been greatly reduced in volume but the organisms of interest have not been segregated from other organism so the signal to noise ratio is till problematic. In addition, further concentration may be needed for detection without enrichment during which one organism is converted to many at the cost of time and delay in decision making.

Several schemes are practical but all involve binding the organisms of interest in a small area such as a microfluidized or nanofluidized channel. This channel or area may or may not be filled with surface activated nanofiber. Utilizing elasto-inertial microfluidics, the viscoelastic flow enables size based migration of larger particles into a non-Newtonian solution, while smaller bacteria remain in the streamline of the blood sample entrance and can be separated. It is tempting to consider surface activated magnetic particles; however, the mechanical manipulation of these particles to achieve the desired small volume is a larger engineering challenge than activating the small surface area. However, any binding geometry that fixes the organisms of interest and any other organisms selected to represent the other microflora in an appropriate small volume can be used.

The surface activation may require numerous active binding sites in close proximity. The mixed DNA primer arrays of SnapDNA are one class of materials. Another class is a cocktail of antibodies for all the organisms of interest.

The binding mechanism may bind all the organism of interest. These organisms may be sufficiently bound that other organisms and materials are selectively removed from the area of binding as clean fluid is passed through the channel.

The motive force to move the cells through the channel can be the mechanical action of fluid flow, electrostatic as the surface of most bacteria is negative, or a size pumping action such as practiced with ferromagnetic particles.

Example Combining Concentration with Partial Cleaning

The challenge of concentrating an extract through filtration is one of surface area and recovery. Get enough surface area to avoid fouling and yet still be able to recover the sample in a small volume. One can use filters aids to artificially increase the surface area of the filter if a special recovery is an option.

Filter aids such as perlite, cellulose, diatomaceous earth, vermiculite and other related porous materials provide depth above a membrane filter where fouling material can be retained. Generally, the desired product from such filtrations is the clarified solution. In the case of pathogen testing there is a unique opportunity. The filter aid can assist in removing water and solutes to effect both some cleaning and concentration. A membrane filter with 0.45 micron pores or smaller will retain all the microbial organisms of interest. The filter cake contains the desired targets and other materials that would have prevented direct membrane filtration.

For 200 ml extractions, a range of filter aid additions from about 0.01 grams to 0.5 grams or more have been found to be useful depending on the level of colloidal solids including dust or clay. An experienced eye can estimate a good level of addition by the intensity of color and opacity of the extract. For standardization, 0.1 grams is a good reference level that works for most samples. It should be noted that increases the levels of filter aid hinder recovery as described next.

In these systems the microbial load would normally be helplessly lost in the small pathways of the filter aid. However, the cells can be lysed while in this matrix and the marker molecules released. The marker molecules can be any soluble molecule that characterizes the targets including nucleic acid, proteins lipids and carbohydrates. These classes can be associated to form other characterizing structures. Although any marker class has the potential to be used, modern molecular techniques prompt the use of DNA or RNA. The amplification and specificity of the polymerase chain reaction (PCR) with or without reverse transcriptase yields tremendous analytical power.

The lysis of the microbial cells can be achieved in situ in the usual ways with a mixture of enzymes, chemical, mechanical and thermal action. An option or a combination of Sodium Hydroxide, Tris, EDTA, SDS, Bead-Beat, GHCl, Chelex100® (Bio-Rad) and boiling has been found to be a good general approach for both Gram-negative and Gram-positive bacteria.

There are three keys to making this concentration and cleaning approach function. First, one must achieve complete lysis and second, one must recover the lysate in good yield. The released markers diffuse much more readily than the intact cells allowing migration form the matrix of the filter cake. Good recovery can be achieved from the filter cake can be achieve by centrifugation and decanting the supernatant. The filter cake can be washed to enhance the recovery of markers but this does increase the volume. Alternatively, the lysate can be recovered through the membrane filter by applying vacuum. The filter cake can again be washed but the cake retains less liquid.

And third, one must use an appropriate filter aid. If the pores are too large or the particles are too large the materials that cause the membrane to foul will be retained in the filter cake and the filtration will be slow or will not allow concentration of the full extract solution. Particle sizes between 10 and 40 microns have been most suitable but mixtures of particles sizes can be used to reduce costs. Care must be taken that the filter aid does not add contamination to the sample. In some embodiments, this is accomplished by utilizing filter aid materials that have been autoclaved. Darcy numbers of 0.3-300 provide a useful metric of to assure performance. Lysate volumes may be larger than desired by this technique. However, the resulting solutions are very amenable to normal nucleic acid concentration techniques. Binding and elution from a silica column is well known. Alternatively, the nucleic acid can be bound to a suitable surface treated probe to collect the nucleic acid. In either case, the resulting smaller volumes will make any subsequent analysis for sensitive due to the increased concentration.

Examples of Screening Detection

With the partially purified organism or organisms of interest bound in a small area or small volume if the cleaning step proves unnecessary, many approaches are available for screening detection for process control. The functional requirements are clearer. First and foremost is that enrichment culture takes substantial amounts of time, and such should be eliminated or reduced in one or more cases. Second, the screening metric may need to be an index suitable for statistical process control. This implies a measurement with many states as opposed to binary 0 and 1. The magnitude of this metric may relate to the extent of deviation from normal operation and therefore the likelihood that an outbreak could occur. Under these conditions, the detection of deviation can be based on the classic rules for control charting. Furthermore, trend detecting rules have the potential to detect problems before significant deviations have occurred. The statistically based Westcard rules provide one basis for trend analysis.

Expressed differently, an index may be needed that tallies the relative presence of beneficial organisms and potential pathogens and aggregates this information in a useful way. As more information is acquired about specific products, the power of big data will come into play. However, at the simplest levels, the aggregate level of potential pathogen is a useful screening tool. The relative balance between potential pathogens and beneficial organisms is a more sophisticated analysis to compensate for seasonal variations that are inherent in many products. It is reasonable to expect to develop indices that are product specific.

The information behind indices is evolving rapidly. The simplest useful index is a ratio of pathogen intensity to a benign organism. These can be generated by many means including classical enumeration with plating, but the classical methods are two slow to meet the functional requirements outlined above. However, given the concentration of the organisms on the cleaning substrate or the concentrated extraction, it is possible to go directly to qPCR in some cases to generate index data.

There are two schemes for generating this type of data at its most sophisticated level. First, one can use a collection of ligands (antibodies, aptamers, or others) that bind and tag all the organisms of potential interest yielding a collection of signals that are multiplexed into a family of useful channels. Alternatively, one can generate an array of specific binding interactions that are analyzed chemometrically to yield a metric. The latter approach will be faster and probably less expensive after the research and analysis is done.

As an example of the first scheme would be a using a mixture of conjugated antibodies to bind to all types of cells of interest. For produce, enterohemorrhagic E. coli, Salmonella and Listeria are of greatest interest. Poultry focuses on Campylobacter and Salmonella. Other industries have other and additional interests. These antibodies can be bound to the organisms bound to the cleaning substrate. The retained tags with either a fluorescent probe or an enzyme provide signal amplification and detection. When the cleaning region as a small enough cross section, the signal from the hundreds to thousands of cells on the cleaning substrate are detectable. Micro and nano fluidization are necessary. However, specific detection protocols can be evaluated at a macro scale using an appropriately instrumented microscope that can be used to measure the signal from a small area where cells have been collected. For speed to market, multiple detectors can be run in parallel or series to generate similar multiple channels of data.

The alternative scheme invokes the lab on a chip concept. By building an array of binding sites, the composition of the samples can be queried. PCR can amplify the contents selectively with a collection of primers. Such detectors can evolve to providing both the screening detection of this step and the ultimate secondary screening. However, it is likely to remain a two-step process due to the economics.

A number of technologies can meet these requirements including but not limited to, for example: 1) Sensors where the vibrational frequencies (which may for example include optical waveguide), impedance, or other properties of a transistor are modified by the binding of the organisms of interest to the sensor. This approach may require that the sensor be built into the surface of the channel. Another technology may include, 2) qPCR where the cells are laid in place and the number of copies of the organisms of interest, or the number of ribosomes of the organisms of interest are estimated. Another technology may include, 3) Use tagged antibodies to light up the organisms of interest so that they can detected on the absorbent surface spectro-photometrically. Enzyme, fluorescent probes, and other materials to amplify the signal can be used.

It may be desirable to avoid confirmation of the presence of one or more specific pathogens during this screening. This is where the trade between LOD and speed to useful information. If the signal is not about three times background, no further action is warranted.

Given that economics may play a role in testing, it should be noted that the sampling approach is compatible with various compositing strategies. Samples can be composited anywhere along the processing path before detection. In various situations, one position will be more advantageous than others. Pooling samples prior to extraction reduces work but reduces the resolution of the results if a positive result is found. However, if the result is negative tremendous savings are achieved. After concentrating and cleaning would allow sampled to be retested if only a portion of the extracted sample is used in a wet pooling approach which can avoid detections costs. Each system needs to be evaluated to determine how to best control costs.

Examples of Confirmation

The confirmation step will be applied when there is reason to suspect that a pathogen is present. The technology in this area is evolving rapidly with many new approaches and efficiencies being developed and introduced. Some will use the techniques suggested for screening with more specific reagents for the confirmation. At present, all the common procedures rely on either molecular biology or ligand binding reactions. Various strategies have been developed to amplify the signal noise ratio and to identify the contaminant to the desired specificity. The desired specificity ranges from simple speciation to identify specific serotypes.

For confirmation, a concentrated sample such as afforded the screening detection above may be included. The concentration may be included due to the small volumes that are compatible with these types of procedures. The organisms, the surface antigens from the organisms, or the nucleic acid from the organisms may need to be extracted from the screening system to the extent that these materials can interact with the reagents of the confirmation procedure. In other words, there is not a priory reason that the detection module could not be engineered for a second round of chemistries. Any one of these materials may contain the information necessary to characterize the contaminant. There are a number of these processes and they are being improved. The choice of approach will be driven by cost, desired specificity and the desired speed.

Many strategies are available to amplify the base signal from these materials that will still be present at only modest concentration in the typical sample. The yield of material from the hundreds to thousands of organisms of interest bound to the screening detector platform is still very small. Amplification based on radio isotopes are largely out of favor but still possible. However, enzyme systems are still in use and new enzymes strategies are still being developed such as those used for ELISA methods or with a luciferase. Fluorescent tags on specific antibodies such as those proposed for the screening determination are less useful for this purpose due the high potential for cross reactivity. However, antibodies of this type are the basis for the serotyping classifications that up until recently has been the standard for characterization. These older serotyping assays may require isolation and growing the organisms.

Increasingly characterization is based on the presence of sequences of nucleic acid. These can be nuclear DNA, ribosomal RNA, or messenger RNA. At the extreme, it is now practical to sequence the entire genome of the organism. Increasing specificity is useful for identification of the source a contamination. However, it also presents a potential liability in the case of an outbreak of illness.

The molecular assays are built around the use of enzymes to replicate sequences of nucleic acid to generate a large enough signal for detection. Various primers are used to select which portions are copied; up to and including the entire genome. For measurement, various fluorescent tags are used. One of the newest techniques utilizes the melting and binding of target material to known sequences to generate a complex matrix of information that can be used chemometrically in lieu of complete sequence data.

Examples of Reporting and Roll Up

Both the screening detection and the confirmation results are reported directly to a sequel database. This reporting may not require human intervention if the included quality assurance standards (positive and negative controls) fall within normal ranges. This avoids transcription and transposition errors. Digital records are more reliable and accurate than manual records.

Both detectors should be part of the “Internet of Things”. This connectivity allows results to be pushed to operators on the floor allowing for the rapid release of product or for the redisposition of product if a potential issue is identified that may need to be addressed. Once the results are in an appropriate database, various users can have customized interfaces providing the information. Some may need to track individual results. Others may be more interested in trends and averages. There may be provided a class of users that may want to aggregate even larger data sets to compare across locations.

There are many platforms available for extracting information from the database. For example, but not limited thereto, Ignition has proven useful in this record as it is open source allowing customization.

In accordance with an aspect of the disclosure, a method for microbial sampling food may include gathering a microbial sampling from one or more food items, extracting microorganisms from the microbial sampling, concentrating the microorganisms, cleaning the microorganisms, tallying a relative presence of the microorganisms and any potential pathogens, aggregating information of a microorganism tally from the tallying of microorganisms into a microorganism report, confirming the microorganism tally, and reporting the microorganism report of the microorganism tally.

In some cases, gathering the microbial sampling from the one or more food items includes sampling, using an aggregating sampler, the one or more food items that include a production lot of produce or meat creating one or more samples that makes up the microbial sampling. The one or more samples may be configured to be processed to indicate if pathogens are present at no greater than a normal background.

In some cases, the method may further include assessing, using an aggregating sampler, a level of cross contamination control to validate or verify a wash process. Gathering the microbial sampling from the one or more food items may include providing an aggregating sampler at a sampling location. The sampling location may be at least one of in a field, at harvest, just after dumping or cutting, in a wash system, or after the wash system.

In some cases extracting includes enriching the microbial sampling, and adding fluid to the microbial sample. In some cases concentrating includes filtering extraction fluid of the microbial sampling using at least one of centrifugation filtering or osmotically filtering. In some cases, cleaning includes binding the microorganisms in a small area including one or more of a microfluidized or nanofluidized channel. In some cases tallying includes using a collection of ligands that bind and tag all the microorganisms of potential interest yielding a collection of signals that are multiplexed into a family of useful channels. In some cases ligands include one or more of antibodies, oligos, aptamers, dendrimers.

In some cases tallying includes generating an array of specific binding interactions that are analyzed chemometrically to yield a metric. The method may further include building an array of binding sites, wherein the composition of the samples can be queried, and amplifying, using a PCR, the contents selectively with a collection of primers.

In some cases confirming includes extracting surface antigens from the organisms or nucleic acid from the organisms from a screening system to the extent that these materials can interact with the reagents of a confirmation procedure. In some cases confirming further includes amplifying a base signal of the microorganism tally.

In some cases, the method may further include use of an index as a surrogate for direct results regarding presence or absence of organisms of interest. In some cases, the method may further include use of a statistical process control for detecting deviations in microbial flora.

In accordance with an aspect of the disclosure, a method of applying aggregating sampling to food items including providing at least one aggregating sampler at one or more sampling locations, and sampling, using the at least one aggregating sampler, a production lot of produce or meat creating one or more samples that makes up a microbial sampling.

In some cases the one or more samples are configured to be processed to indicate if pathogens are present at no greater than a normal background. In some cases, the one or more sampling locations includes at least one of in a field, at harvest, just after dumping or cutting, in a wash system, or after the wash system. The method may further include assessing, using the aggregating sampler, a level of cross contamination control to validate or verify a wash process.

In accordance with an aspect of the disclosure, an apparatus for microbial sampling, including means for gathering a microbial sampling from one or more food items, means for extracting microorganisms from the microbial sampling, and means for concentrating the microorganisms, means for cleaning the microorganisms, means for tallying a relative presence of the microorganisms and any potential pathogens, means for aggregating information of the microorganism tally into a microorganism report, means for confirming the microorganism tally, and means for reporting the microorganism report of the microorganism tally.

In accordance with an aspect of the disclosure, an apparatus for microbial sampling, includes at least one processor configured to generate control signals for controlling gathering a microbial sampling from one or more food items, extracting microorganisms from the microbial sampling, concentrating the microorganisms, cleaning the microorganisms, tallying a relative presence of the microorganisms and any potential pathogens, aggregating information of the microorganism tally into a microorganism report, and confirming the microorganism tally, and a transmitter configured to transmit the microorganism report of the microorganism tally. In some cases the apparatus may further include an aggregating sampler configured to gather the microbial sampling.

In accordance with an aspect of the disclosure, a non-transitory computer readable medium for microbial sampling having instructions stored thereon for gathering a microbial sampling from one or more food items, extracting microorganisms from the microbial sampling, concentrating the microorganisms, cleaning the microorganisms, tallying a relative presence of the microorganisms and any potential pathogens, aggregating information of the microorganism tally into a microorganism report, confirming the microorganism tally, and reporting the microorganism report of the microorganism tally.

In accordance with an aspect of the disclosure, a method for sampling food including concentrating microorganisms and removing interference, tallying a relative presence of the microorganisms and any potential pathogens, and aggregating information of the microorganism tally into a microorganism report. In accordance with an aspect of the disclosure, a system capable of implementing one or more of the novel aspects discussed in this application disclosure.

In accordance with an aspect of the disclosure, a microbial aggregating sampler, including a covering including a microbial sampling material with a pocket formed in the covering to receive an appendage or a tool for handling of the covering.

In some cases, the covering includes an attachment feature formed in the pocket to receive the tool. In some cases, the attachment feature includes one of a hole formed through the covering, a loop positioned within the pocket to receive an end of the tool there through, and a tab positioned within the pocket for an end of the tool to attach thereto.

In some cases the covering includes a sheath formed in the pocket to receive a digit of an appendage. In some cases the pocket is formed through the covering such that the appendage or the tool for handling the covering extends through the covering. In some cases the covering is completely formed from the microbial sampling material. In some cases the covering includes two sheets attached to each other to form the pocket. In some cases the covering includes a single sheet folded and attached to itself to form the pocket.

Other Applications Beyond Bacterial Testing

The aggregating sampler can be used to sample for additional analytes beyond bacteria including yeast, molds, viruses, allergens, toxins such as aflatoxin, marker indices or particulates such as dust. The commonality is the surface presence of the analyte at low levels that can be concentrated. The basic process is the same with the same key steps. The biggest differences will be in detection strategy, but these strategies are well known by those who study these analytes.

Monitor marker indices can be useful for evaluating and comparing processes. The pathogens are generally present as such low levels that heroic numbers of samples would be required for comparison. Often Aerobic Plate counts, generic E. coli and enteric bacteria are used in this way. However, various genes and PCR techniques are replacing these broad categories of bacteria and provide a more precise comparison. The gene coding for bacterial flagella is an example. The gene coding for Hemolysin is another. It is sufficient to identify a target and create a probe to add a new index.

### An End-to-End Example

To illustrate the invention, we describe here the application of the invention to testing frozen beef trims for the presence of human pathogens. Given the novelty of this approach, it does not have regulatory approval. As such, adjustments can be expected to comply with regulatory requirements. Nevertheless, the principles described should be useful.

To prepare for sampling a lot of beef trims, various supplies need to be prepared including the bagged and bar-coded sampling sleeves, the heating system, a scale, and electronic devices for documenting the tracking of samples. A trained operator with appropriate gloves and cold room clothing is also needed. A working area to facilitate the sampling is helpful. And finally, a work order prepared in the computerized sample tracking program that requests that 5 boxes be sampled.

As the process starts, 5 fifty-pound boxes are transferred from the −20 degrees Celsius freezer to a 4 degree tempering area for testing. After tempering for a short time, the operator scans the bar-code on the bag of the first sleeve using the application on his phone assigning it to the first sample. The peel off label from the sleeve bag is attached to the box linking this box to the sample in the event that follow up sampling or other action is warranted after the process is complete. And finally, the box and inner wrapping are opened exposing the beef trims for sampling.

The sampling sleeve is slid over the heating element in this case a channeled silicon pad with metal reinforcing frame and handle, preheated to 40 degrees Celsius with circulating water with ethanol as an antifreeze. The wrapped probe is run slowly over the surface of the beef trims sullying the sleeve with surface liquid from the trims. This surface liquid is where pathogens if any are expected given that the interior of the trims are essentially sterile with regards to pathogen presence. This process continues until the sleeve is well colored ensuring a good sampling. Care is taken to access any accessible niches in the block of trims and reaching down the sides of the block as the packaging permits. This process is timed and is required to take at least 3 minutes.

After sampling, the sleeve is removed from the sampling probe and returned to the original bag without contacting the sleeve to avoid contamination. The probe is sanitized with alcohol to limit cross contamination risk. The process is repeats for samples two through five. All five cartons are resealed and returned to the lot with their barcodes visible should they be needed. This sampling is considered non-destructive as the quality of the product is unchanged for its intended use.

At this point the bar-coded samples are taken to the test site where they are weighed to ensure that sufficient material has been extracted from the sample. The weight of sleeves in bags is consistent enough that a constant tare weight provides sufficient precision for this determination. Samples that are underweight are not considered valid for negative findings and need to be replaced. However, a positive result for any sample is deemed informative.

The aspects described herein can be used in process utilizing a Sample-Treat-Analyze-Report (“STAR”) process. With weights attached to the sample record, the extraction procedure is performed within a STAR analyzer for use with the STAR approach, the analyzer extracts and concentrates the sample sufficiently for direct molecular determination of the presence or absences of pathogens, generally E. coli O157 for beef. The STAR process can utilize the filter aid and DNA purification technique to avoid the delays of a cultured method. These results are reported to the FSQA data management system to allow for the proper disposition of the lot. Pathogen positive lots need to be directed to commercial cooking processes. It is appreciated that the STAR approach can incorporate any of the various processing features described herein, in whole or in part, as well as variations thereof.

### An Additional End to End Example

The testing of produce, particularly items destined for the fresh cut market has become increasingly important. This pressure is especially important for Romaine lettuce and baby leaf spinach. We elaborate here the application of the invention to spinach.

During harvest, an aggregated sampler, a sheet of sterile non-woven fabric, is placed in the cowl of the harvester elevator belt where it will be impinged by the product during harvest. The cowl moves from bin to bin until a trailer load of 12 bins has been collected. During the time that trailers are being exchanged, a new sheet is installed to sample the surface of the product in the next trailer. The attachment can be performed variously but large clips have proofed to be the easiest. These clips can be mounted directly to the cowl which can be raised and lowered for access.

The sheets are refolded and returned to their original bag and the bar code is scanned to associate the sheet with the trailer. The rebagged sheet will travel with the driver to the processing plant. Generally, two trailers are pulled in tandem meaning the driver will convey two sheets to the receiving area where FSQA will proceed with its normal receiving processes and initiate cooling as appropriate.

FSQA will transfer the sullied sheets to the laboratory where the barcodes will be rescanned to link the pending analysis to the record of the trailer. The sullied swab is extracted with suspension buffer using a stomacher. Other devices can also be used such as a sonic bath or sonic probe. Generally, 150-200 mls of Phosphate Buffered Saline with 0.01% mild surfactant (PBS-T), or normal saline with 0.01% mild surfactant is used.

Baby leaf spinach can be especially dirty carrying large amounts of soil and in some cases diatomaceous earth applied for insect control in organic production. Generally the filter aid approach can handle this extraneous matter but, in some cases, a low speed centrifugation is used to sediment these large particles to facilitate filter concentration. An experienced technician can readily identify the exceptional samples.

At this point 0.1 g of filter aid is added to either the decanted sample or the sample decanted after centrifugation and mixed gently. Extra is filter aid can be included to speed the filtration process for those samples containing more matter. The optical density those samples required extra filter aid generally exceed OD₆₀₀ 1.5.

The filter aid slurry is filtered through a 0.45 micron filter with vacuum. This takes less than 3 minutes and yields an almost dry filter to cake which contains the bulk of the organisms or interest. This filter cake is transferred to a new vessel, usually a 5 ml tube and any dirt sediment as added to the sample.

The combined filter cake and dirt sediment if any are treated with lysis buffer to yield a lysate suspension which is centrifuged and decanted. This lysate is put through a commercial DNA clean up procedure (e.g., QIAGEN, ThermoFisher, etc.) to yield a clean and concentrated DNA sample representing the bacteria from the original sample. This concentrated sample is suitable for PCR testing for the presence of pathogenic E. coli or Salmonella. This whole process routinely takes less than two hours.

Example Systems/Methods of Sample Treatment

In yet another embodiment, the invention pertains to systems for processing a sample medium to extract a purified, concentrated sample suitable for immediate testing without requiring any enrichment, preferably within two hours or less. Such systems can include an extraction container, such as an extraction bag (i.e. sealable sampling bag), in which the sampling medium is disposed and to which an extraction buffer fluid is added. The extraction buffer can optionally be heated to promote extraction of target molecules from the sample medium. The system can further include a filtration module that can include one or more filters. In some embodiments, the filtration module can instead utilize a sample filter cup, including a standard off-the-shelf sample cup. Any suitable filter aid materials can be used. An in situ lysis buffer can be added to promote lysis of target DNA from molecules trapped within the filtration module. Optionally, the in situ lysis buffer can be heated to speed up the process. The filtered lysed fluid sample can then proceed into a binding/mixing vessel container where the sample fluid is mixed with a binding buffer liquid that binds to the target DNA. Any waste products from the filtration module or binding/mixing vessel can be sent to a waste reservoir. After binding, the fluid sample is then transported to a nucleic acid (NA) affinity column, to which an elution buffer is added to facilitate elution of a concentrated sample from the column. Optionally, the elution buffer can be heated to speed up the process. Additionally, any waste products from the NA affinity column can be emptied to the waste reservoir. The eluted sample can be collected by a sample vial, which is then tested for detection of the target. Optionally, the sample vial or associated container can be held in vacuum so as to facilitate flow of eluted sample from the column without requiring pressurization of the column or pumping.

In some embodiments, the above system includes various containers (e.g. bags, buffer reservoirs, chambers/vessels for mixing, affinity column) that are interconnected by a network of conduits (e.g. pipes, tubing). In some embodiments, the networks includes one or more types of valves (e.g. one-way valves, actuatable valves, pinch valves) to facilitate controlled fluid flow of fluid sample and buffers through the system. In some embodiments, the system further includes one or more injection or pumping systems to facilitate controlled fluid flow through the system. In some embodiments, the system includes one or more air vents (e.g. inlets, outlets, bubble vents) to facilitate free flow of fluid through the system. In some embodiments, any of the valves, vents, injection or pumping system are controlled by one or more control units that provide automated actuation of any of these elements in a coordinated sequence to facilitate controlled flow of fluid sample through the system to produce the purified, concentrated system. In some embodiments, the system includes a centralized control unit.

In some embodiments, the system is configured as a flow through system so that no mechanical transfers are required. In some embodiments, the use of heated buffers speed up the various reaction and processes described herein. In some embodiments, the system can include readily removable components so that any surface contacting the fluid sample can be removed/replaced between samples. For example, the injection unit that facilitates extraction of fluid sample from the extraction bag to the filtration unit can be readily removable/replaceable between samples. Further, the filtration module, binding/mixing vessel, and NA affinity column and fluid pathways therebetween can be incorporated into a cartridge that is readily removable/replaceable between samples.

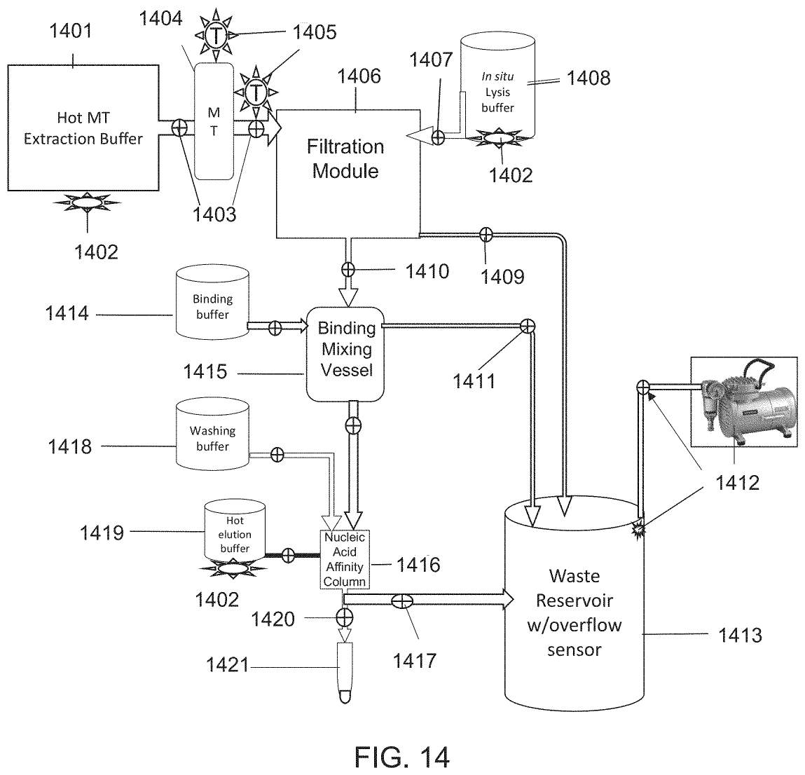

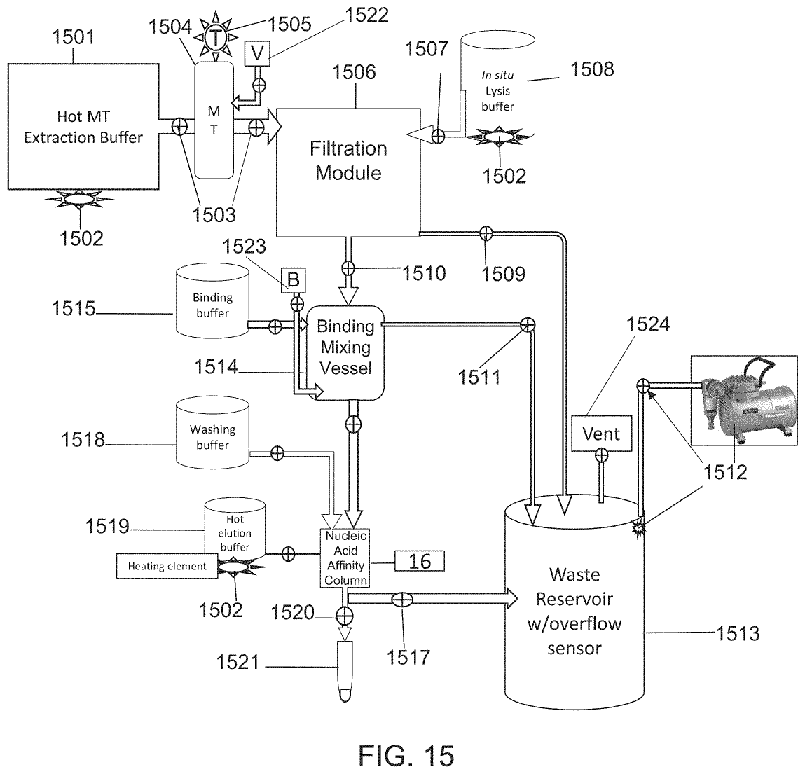

Such systems can be further understood by referring to the examples in FIGS. 14-15. FIG. 14 shows system 1400, which includes extraction bag 1404 that receives a sampling medium (e.g. aggregate sampler, MicroTally® swab, filter aid materials, etc.), an extraction buffer reservoir 1401 having a heater 1402 with associated thermo-controllers, and pinch valves 1403 actuated by a central control unit, known as a programmable logic controller (PLC), so as to control flow of extraction buffer into the extraction bag and flow of extracted sample to filtration module 1406. An injection system 1405 controls flow of extraction buffer from the reservoir 1401 to the extraction bag 1404 and flow of extracted sample fluid from the extraction bag 1404 to the filtration module 1406. The filtration module 1406 can include one or more filtering features that filter according to any desired protocol. In the embodiment shown, the filtration module 6 is a 25 mL, 0.45 μm filtration system. It is appreciated that in other embodiments, the capacity and degree of filtering of the filtering module could be scaled according to the type and volume of sample. In this embodiment, an in situ lysis buffer contained in a lysis buffer reservoir 1408 is fed into the filtering module 1406 by an actuator controlled valve 1407. The in situ lysis buffer can include any suitable lysis materials/fluids, which can further be heated (e.g. pre-heated to 70° C.) by a heating element 2. Lysis can include alkali compounds or any suitable compounds, and can further include application of heat. Any waste material is fed into the waste reservoir 1413, which is controlled by valve 1409.

After filtrating of the sample fluid in the filtration module 1406, the filtered nucleic acid (NA) in situ lysate is fed into the binding/mixing vessel 1415, which is controlled by valve 1410. A binding buffer from a binding buffer reservoir 1414 is fed into the binding/mixing vessel 1415 by another control valve. In the binding/mixing vessel 1415, NA precipitation forms after sample lysate mixes with the NA binding buffer. In this embodiment, a delta pressure actuated valve is used to build up a negative pressure for the binding mixing vessel, that lets both in situ lysate and binding buffer run into the vessel to prepare the nucleic acid to bind within the purification column. Any waste material is fed into the waste reservoir 1413, which is controlled by valve 1411.

The sample then flow via another actuated valve into a NA affinity column 1416. A washing buffer from washing buffer reservoir 18 can be fed into the affinity column via another valve controlled conduit. A preheated NA eluting buffer from reservoir 1419 is injected via actuated valve into the NA affinity column. In some embodiments, the elution buffer can be heated by heater 1402 to speed up the process. Any waste material is fed into the waste reservoir 1413, which is controlled by valve 1417. The filtrate waste reservoir 13 collects the sample extracts' filtrate, nucleic acid (NA) isolation filtrates, and NA column washes filtrate/waste. A sample elution control valve 1420 controls flow of the final concentrated, purified NA sample into a NA elution collection vial 1421. In some embodiments, the vial is enclosed in a vacuum to promote flow through the affinity column and into the vial 1422.

FIG. 15 shows another example system 1500 for producing a purified, concentrated sample from a sampling material, without requiring any enrichment. The noted features 1501-1521 correspond to the same or similar features 1401-1421 in the embodiment of FIG. 14; however system 1500 further includes various vents to facilitate flow of the fluid sample through the system. As shown, system 1500 includes an air vent 1502 from extraction bag 1504, which controls flow of air to facilitate flow of fluid sample from the extraction bag into the filtration module 1506. In this embodiment, air vent 1502 is a controlled air inlet into the extraction bag. In some embodiments, for example, where the extraction bag is the sampling bag, this vent is provided by merely opening the sealable top of the bag. System 1500 can further include a bubbling vent 1523 on the binding mixing vessel 1504 to facilitate mixing of the fluid sample with the binding buffer liquid. The waste tank 1513 can further include air vent 1523 to facilitate flow of waste into the tank. It is appreciated that in other embodiments, one or any combination of these vents could utilized, or that various other types of vents could be used or located along various other locations (e.g. reservoirs, conduits, etc.) to promote flow of fluid sample through the system. In some embodiments, the network of fluid conduits and any associated vents and valves are configured to facilitate flow of fluid sample through the entire system by a single vacuum source 1512.

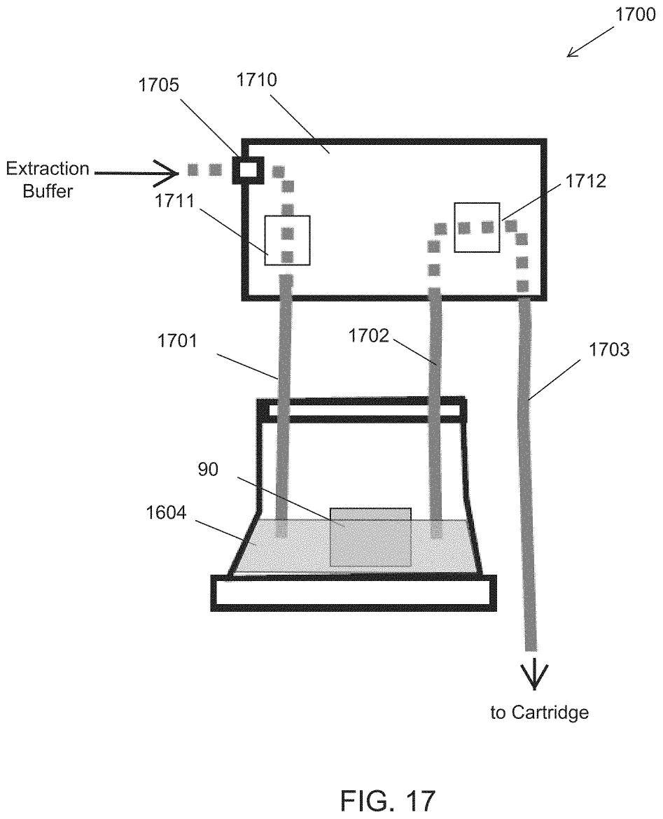

In an exemplary method, which can be further understood by referring to FIGS. 14-15, the sample treatment process involves an initial extraction of fluid sample form a sampling member (e.g. MicroTally® swab) or filter medium within an extraction bag (e.g. sampling bag) by use of an extraction buffer (e.g. typically ˜150 ml of heated extraction buffer). In some embodiments, pretreatments may be performed to remove color, reduce viscosity or remove excessive soil. In some embodiments, any filter materials (e.g. special filter aid blend) can be either added to the extraction bag or metered in during the filtration process. A siphon tube or vent at the bottom of the extraction bag feeds the extraction buffer into the filter chamber by a mixture of gravity and vacuum (see FIG. 17). The vacuum is sufficient that gravity is not required but can aid in assuring complete transfer of the extraction. The resulting filter cake can be partially dried by air flow. The valve below the filter is closed and heated lysis buffer (e.g. 5-10 ml) is fed into the filter chamber by drawing a vacuum on the top of the filter chamber. If agitation is desired, an air bubbler (not shown) can be used with vacuum to mix the filter materials/lysis buffer slurry. The lysate is then drawn into the mixing/blending vessel by the vacuum. The lysate is then mixed with a binding buffer, optionally an air bubbler agitation provides additional mixing. This mixture is then drawn into the NA affinity column. A washing buffer is drawn into the column and then the purified, condensed nucleic acid is eluded into 50 μl of buffer and dispensed into a test tube ready for molecular analysis.

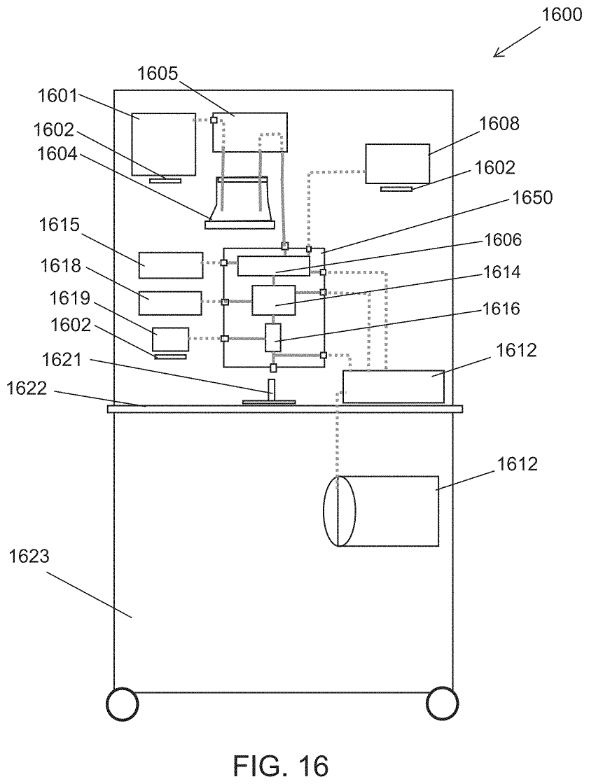

During the process various valves are open and closed to direct vacuum to the appropriate vessels and allow flow. The valve can be a binary valve (e.g. on/off), one-way valves or any suitable valve. In some embodiments, one or more valves can be reusable actuators for tubing pinch valves, which simplified the design and reduces costs. In some embodiments, the pinch valve is provided to interface with tubing on a cartridge inserted into the system (see FIGS. 16-18).

In some embodiments, the system utilizes one or more pretreatment processes, which can include any of the following: an oil extraction of a lipid layer forms on the surface of the extraction; an enzyme treatment to reduce viscosity by hydrolyzing hydrocolloids such as pectin; and a color shifting treatment to reduce pigment interference in molecular assays.

In some embodiments, the system components comprise a manifold or network that requires only a single vacuum source for moving the fluids through the system. The vacuum source may be integrated with the device or may be external and connected to the device. Accordingly, in some embodiments, the network can include binary valves (e.g. tubing pinch systems) that leave the actuators in the device for reuse. In some embodiments, the system can include one or more pumps or injectors to facilitate fluid flow through the network.

FIG. 16 shows another example flow through system 1600 that is mounted on a mobile cart to allow for rapid sample treatment and testing on-site. System 1600 includes system features 1601-1621 that correspond to the same or similar features 1401-14021 in the embodiment of FIG. 14; however system 1500 further includes a mobile-cart 1623 having wheels and a shelf 1622 to facilitate sample elution into the test tube 1622, and a removable sample injection system 1605 and removable cartridge 1650 that includes the filtration module 1601, binding/mixing vessel 1614 and affinity column 1616 as well as various tubing, valves and connectors. These removable portions allow the surface/conduits that contact the sample to be readily replaced with a new injection system 1605 and cartridge 1650 of like construction between samples. Accordingly, each of these replaceable portions can include mounting hardware and fluid-tight coupling connectors to connect the tubing to the piping of the system. Notably, the cartridge need only be sized to accommodate a single sample, whereas the reservoirs of the system holding the various buffers can be sized to provide buffers for many samples.

FIG. 17 shows a detail view of the removable replaceable sample injection unit 1700 (corresponding to unit 1605 in FIG. 16). This unit includes a body 1710 that includes a buffer inlet coupler 1705 that connects the extraction buffer conduit to a buffer tubing line 1701 that is inserted into the extraction bag 1604 and a control means 1711 that controls flow of buffer into the extraction bag 1604, in which the sampling medium 90 is contained. Injection unit 1700 further includes a sample inlet tube line 1702 and a sample outlet tube line 1702 and a control means 1712 that controls flow of extracted fluid sample from the extraction bag through tubing lines 1702/1703 into the cartridge. In this embodiments, the tubing lines are flexible tubing so as to be readily insertable into a flexible extraction bag. It is appreciated that various other types of tubing or conduits could be used. The control means could include a powered pump or injection mechanisms, or could include a valve connection to one or more vacuum sources.

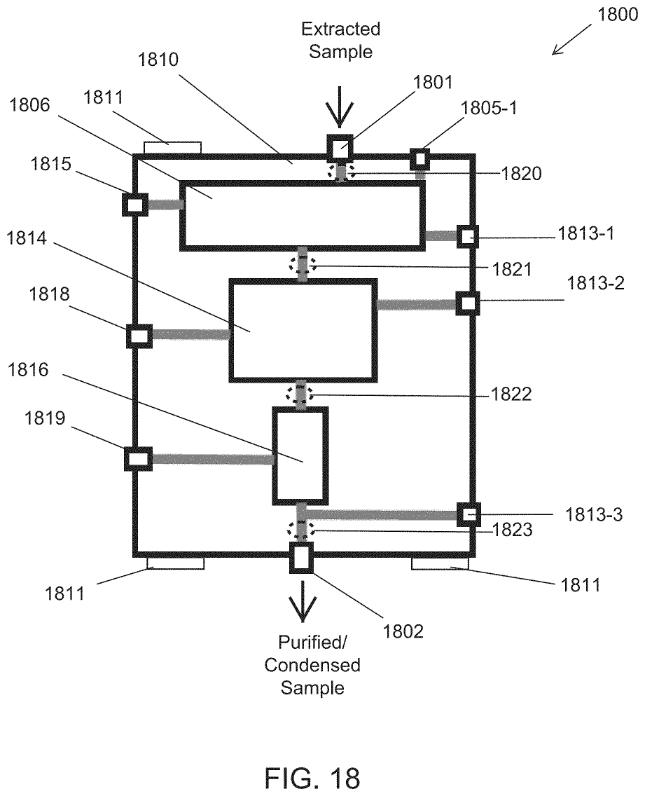

FIG. 18 shows a detail view of the removable cartridge 1800 (corresponding to cartridge 1650 in FIG. 16). In some embodiments, this cartridge is readily removable and disposable. The cartridge includes a planar body 1810 that includes various vessels or chambers, a network of conduits (e.g. tubing, piping) that interconnect the vessels and connect to the various buffer and waste conduits. In particular, the cartridges includes a sample inlet coupler 1701 that receives the extracted sample and feeds the sample fluid into the filter module 1806 through valve 1820. In some embodiments, the filter module includes filter aid material so that the filter module can filter the fluid sample and any waste fluid is directed to the waste conduit/reservoir. In other embodiments, the filter module can be configured to receive filter aid materials that previously filtered a fluid sample, for example a filter from within a sample cup. The in situ lysis buffer is fed into the filter module through fluid coupler 1815 and any waste is removed via fluid coupler 1813-1. The filtered fluid sample is then fed into the binding/mixing vessel 1814 through valve 1821. The binding buffer is fed into the binding/mixing vessel 1814 through fluid coupler 1818 and any waste is removed via fluid coupler 1813-2. The mixed sample is then fed into the NA affinity column 1816 through valve 1822. The washing buffer is fed into the affinity column through fluid coupler 181 and any waste is removed via fluid coupler 1813-3. The purified/condensed fluid sample is then dispensed via valve 1823 through outlet 1802. It is noted that valves 1820, 1821, 1822, and 1823 can be any suitable type valve actuated by a user, pressure, a central control unit or any suitable means. In some embodiments, the cartridge includes the actuatable valves thereon. In some embodiments, the cartridges includes merely flexible tubing and the valves are provided on a cartridge interface of the system, for example, pinch valves that engage against the tubing on the cartridge. In some embodiments, the buffers or waste conduits can include valves. In the embodiment shown, the buffer or waste conduits are merely connected to fluid-tight couplers that removably connect to buffer or waste conduits on the system, which can include actuatable valves that are actuated by the user, pressure, or preferably an automated control means (e.g. a central control unit). The cartridge can further include any suitable mounting hardware (e.g. latch, lock, clamp, magnet, etc) so as to removably mount the cartridge to the system, for example, mounts 1811 that interface with corresponding features on the system to secure the cartridge in place with the cartridge fluidically coupled the system interface.

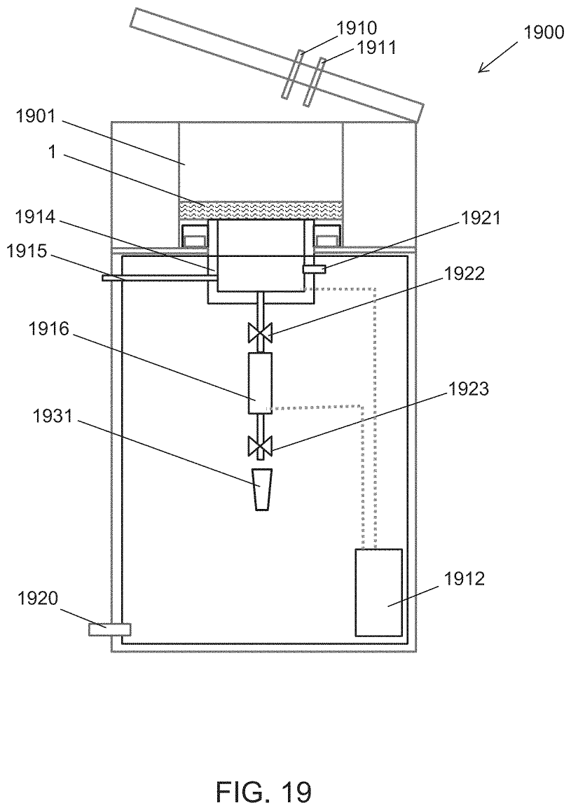

FIG. 19 shows another embodiment of a system 1900 that is configured to receive a filter cup 1901 and perform same/similar processing steps on the sample as discussed in previous embodiments in FIGS. 17-18 and can include various corresponding components (e.g. couplers, valves, conduits, reservoirs, etc.) such as any of those described herein. In this embodiment, the system 1900 can receive a separate filter cup 1901 having filter aid materials 1 within for filtering the fluid sample and trapping target molecules in the filter aid materials. In some embodiments, the filter cup previously filtered the fluid sample at another location and is subsequently placed in a receptacle or interface of the system 1900 for preparation of the sample. In other embodiments, the filter cup can be placed in the system and the fluid sample can be filtered while disposed in the system. In the latter approach, the waste fluid from filtering can be directed to a waste reservoir 1912 through a waste conduit. Any waste fluid from any of the steps herein can be directed to the waste reservoir 1912 shown. In some embodiments, the filter cups can be standard off-the-shelf filter cups. Some such filter cups have an open flow through design such that the bottom can be fitted onto an open conduit for sample preparation by the system. Other such filter caps include an integrated valve at bottom, which can be coupled with a correspondingly sized conduit. Thus, the system can be configured for use with either filter cup design, or can include one or more adapters so as to be compatible with various types of sample cup designs.

In system 1900, the filter cup 1901 filters the fluid sample. Typically, the filter cup is used to filter the sample and reject the bulk of waste water from the fluid sample before the sample cup placement in the system. This cup is then transferred to the system with the filter aid materials (e.g. filter cake) therein. System 1900 can include a lid that closes over the sampling cup and the lid can include one or more inlets coupled with corresponding conduits that transport processing buffers from designated reservoirs. In this embodiment, the lid includes inlet 1910 for lysis buffer and inlet 1911 for neutralizing buffer so as to dispense the buffers into the filter aid materials to lyse the target molecules trapped within the filter aid materials. In this embodiment, this upper region of the system 1900 is open to air. After lysing, a vacuum can then be applied to the system through vacuum port 1920 to suck the fluid containing DNA from the lysed target into the binding/mixing vessel 1914. Washing buffer can be injected into the vessel through conduit 1915 and mixed through any suitable means (e.g. agitation, air bubbler, etc). The vessel can be vented to atmosphere through vent 1921. By actuation of valve 1922, the washed fluid containing DNA from the lysed target can be sucked into the nucleic acid affinity column 1916. Binding buffer is added to allow nucleic acids to stick to column and elution buffer is then added to elute/release the DNA from the column for elution into a test tube 1931 (e.g. snap top vial) by actuation of valve 1923. The vial can then be transferred to an analytical device for testing.

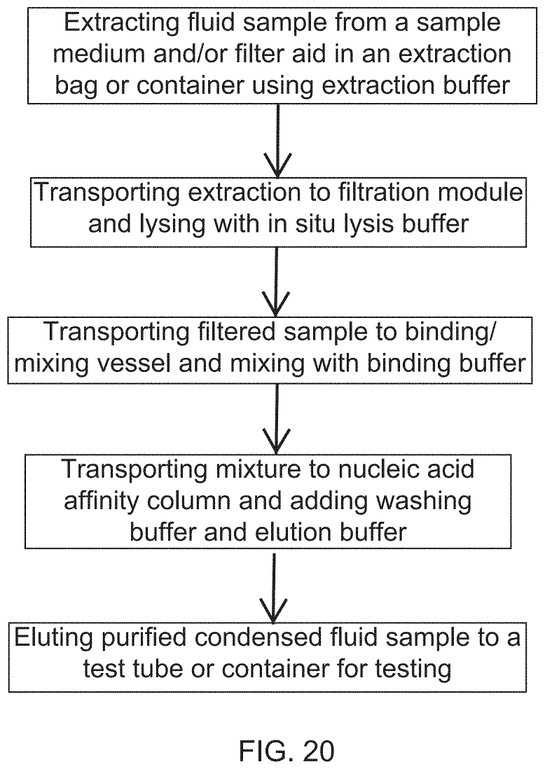

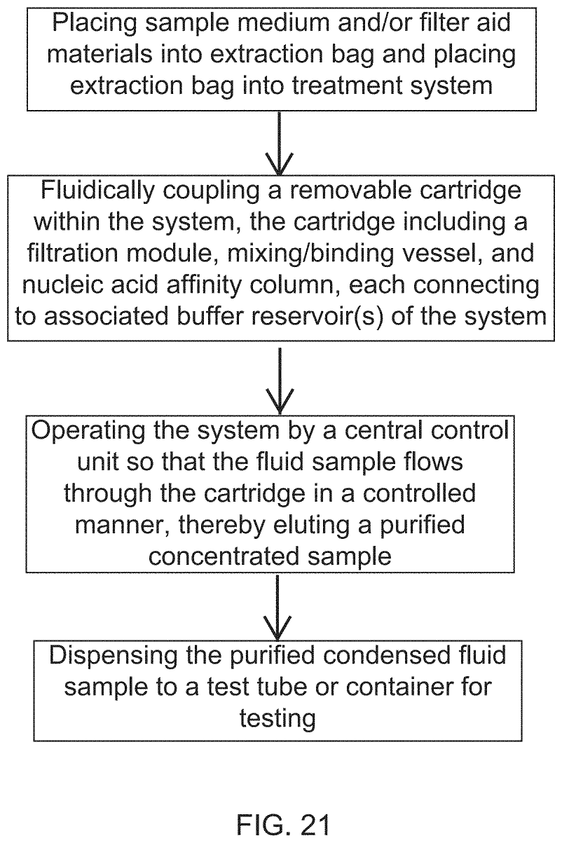

FIGS. 20-21 show exemplary methods of sample treatment utilizing the concepts and systems described herein.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Alternative Modes of Concentration by Filtration

Although the preferred method of use for speed is to extract a sample, concentrate by filtration (also referred to as a “T-factor” approach), and lyse in place, other modes of use can be used when more sensitivity is needed or when time is less important than minimizing labor or technical requirements.

In one aspect, a more sensitive approach with only a minor increase in time to result is obtained by a short incubation of the sample in filter. In some embodiments, this enrichment is affected by adding a small amount of enrichment media to the filter aid materials (e.g. filter cake) on the filter. This process can be accelerated by pre-warming the enrichment media. It is recognized that this enrichment could also be carried out by transferring the filter cake to a second vessel, however this could increase the required manipulations and therefore increase costs. Typically, this small amount of enrichment would be several volumes of the filter cake, generally 10-30 mls. The smaller the volume, the more concentrated the resulting enrichment will be in the same amount of time. Most molecular method have optimal effectiveness at around 1000 cells/ml because only a few microliters are used in the assay. By performing a short enrichment in situ around and in the filter cake, the enrichment can easily be reconcentrated by a standard filter concentration process (e.g. T-factor process) whereby the nuclei acid are concentrated into a suitable amount, such as 50-100 μl where less than 100 copies are reliably detected. Enrichment times will depend on the lag phase and will vary but 2-4 hours is amble. Times as short as one hour might be sufficient when less enrichment is required.

Another alternative approach is to extract the sample into a warm enrichment media. This will require more enrichment media than the previous approach, but this is not a large cost when compared to eliminating the extra manpower of a second concentration step. Typically, the required enrichment will still be short because the ultimate concentration is the same. Thus the small number of cells in the sample still need only grow to the same extent, which will be about the same amount of time. In this approach, the enrichment is concentrated as previously outlined for a standard filter concentration process.

Still another alternative approach completely avoids the in situ lysis in the filter cake. In this approach, the sample is extracted in the usual manner and subjected to a standard filter concentration process. The filter cake can be enriched in place or transferred to a secondary container. A suitable amount of enrichment media is added and the sample allowed to enrich to directly achieve suitable levels, for example, 1,000 copies/ml. This enrichment will normally be between 4 and 8 hours depending on the lag phase and organism. This approach trades time for labor and technology and removes inhibiting materials better than simply diluting a sample for enrichment.

Step-by-Step Description of Flow-Through Process

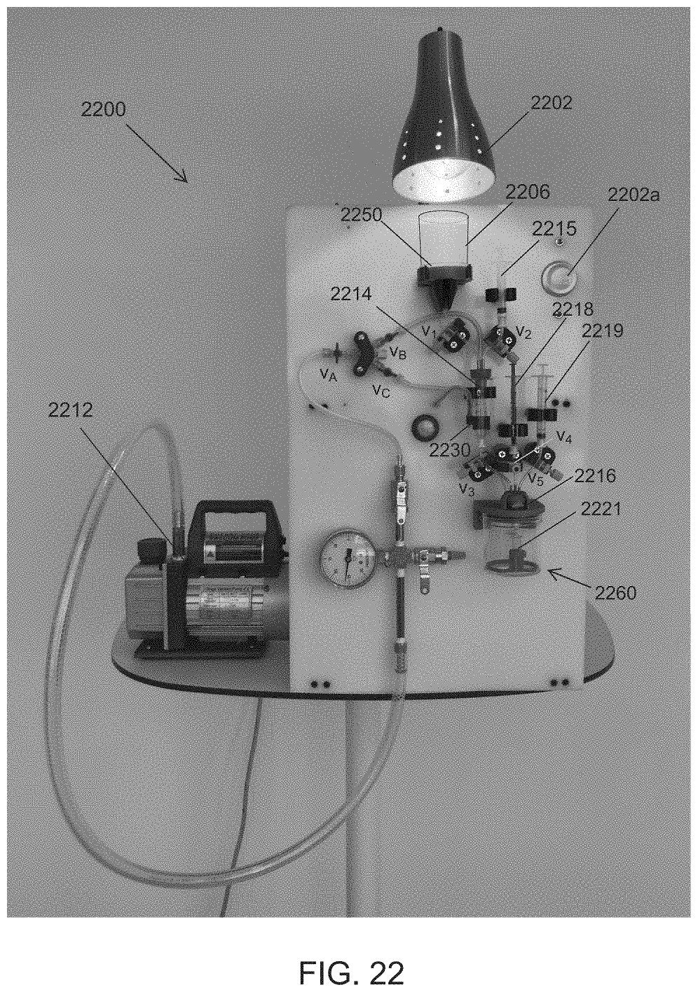

FIG. 22 shows another example flow-through sample treatment system 2200 that allows for rapid sample treatment for testing on-site. System 2200 includes various system features 2202-2219 that correspond to the same or similar features 1402-1419 in the embodiment of FIG. 14; however the flow-through setup system 2200 starts with positioning of a filter cup 2250 that includes the sample and which can include filter aid materials (e.g. filter cake) as described herein. The system can include an interface for coupling with the filter cup, which can include a funnel for transitioning a bottom opening in the filter cup with fluidic conduits (e.g. ⅛″ OD polymer tubing) of the system. A heater 2202 can be disposed above the filter cup (e.g. incandescent bulb) and can include modular positioning and an adjustable switch 2202a. Once positioned and fluidically connected to the fluidic network of conduits and valves (e.g. pinch tube valves) in system 200 the flow of sample solution from filter cup 2250 is controlled by application of vacuum pressure through one or more air tubes coupled to one or more network elements and by selective opening of one or more valves. Vacuum valves Va, Vb, Vc allow the vacuum pressure to be selectively applied within the network to facilitate controlled fluid flow therethrough. For example, opening of valves Va, Vb applies vacuum pressure to binding/mixing vessel 2214 so as to suction the sample solution from filtration cup 2250 through a conduit and open valve v1 into the binding/mixing vessel 2214, in which the sample can be mixed with an injected buffer by agitation motor 2230 attached thereto. Binding and/or neutralizing buffer can be injected from buffer reservoir 2215 (e.g. syringe). After sufficient mixing, application of vacuum in collection cup 260, by opening valve VC, suctions the mixed sample solution into the DNA affinity column 2216. Buffers from the respective washing buffer reservoir 2218 and elution buffer reservoir 2219 (e.g. syringes) can be injected into the affinity column 2216 to wash and elute the sample. Then, the sample solution along with any DNA from the target analyte, is eluted into the sample vial 2221 within the collection cup 2260.

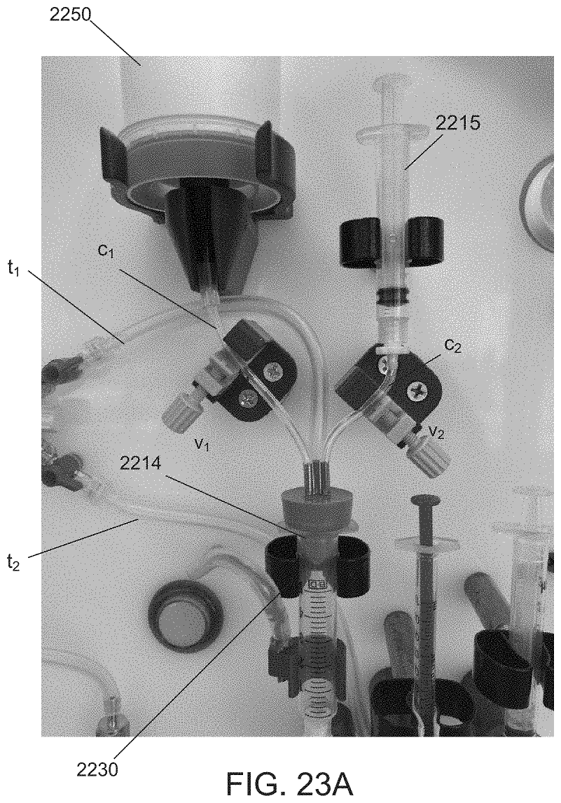

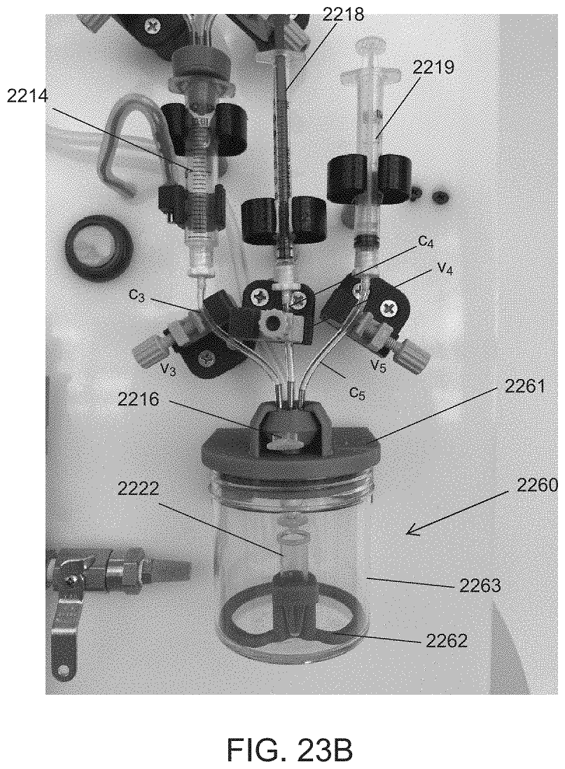

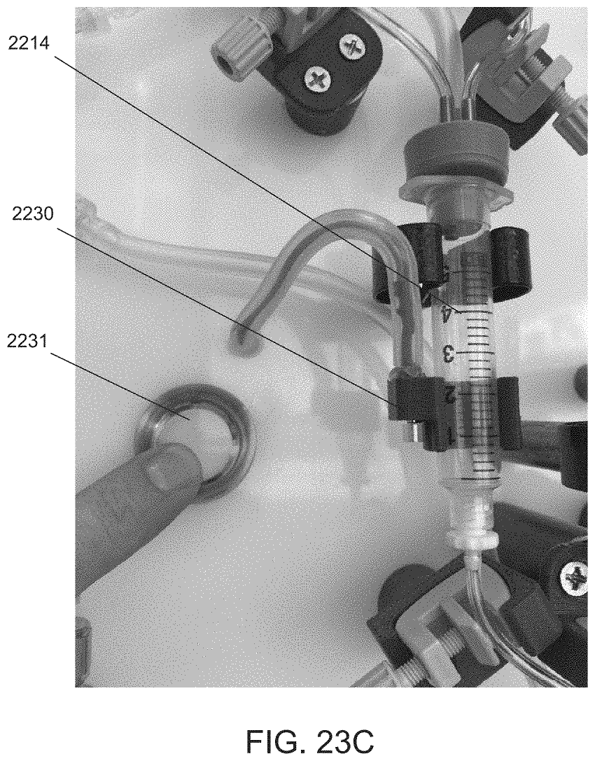

FIGS. 23A-23C show detail views of portions of the flow-through setup system 2200 in FIG. 22. As shown in FIG. 23A, valve v1 opens a fluid conduit c1 connecting filtration cup 2250 with the binding/mixing reservoir 2214 and valve v2 opens/closes fluid conduit c2 that connects buffer reservoir 2215 with binding/mixing reservoir 2214. Similarly, as shown in FIG. 23B, valve v3 opens/closes the fluid conduit c3 that connects the binding/mixing reservoir 2214 with the affinity column 2216, valve v4 opens/closes the fluid conduit c4 that connects buffer reservoir 2218 with affinity column 2216, and valve v5 opens/closes the fluid conduit c5 that connects elution buffer reservoir 2219 with affinity column 2216. The collection cup 2260 includes a lid 2261 that seals atop the collection cup container 2264 and supports the affinity column 2216 therein. The collection cup 2260 can further include a vial stand 2262 for supporting the sample vial 2222 beneath the affinity column to facilitate elution from the column into the vial. As shown in FIG. 23C, the user can press agitation button 2231 to activate agitation motor 2230 disposed on the binding/mixing reservoir 2214.

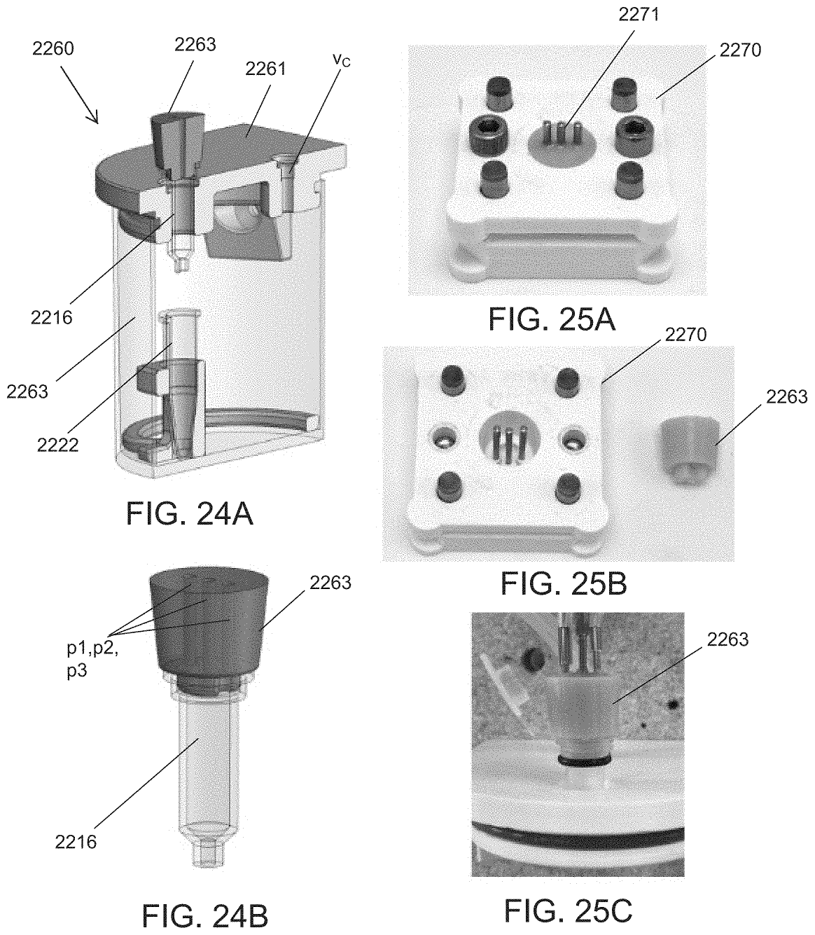

FIGS. 24A and 24B show the collection cup 2260 and DNA affinity column 2216. As noted above, the collection cup includes a lid 2261 that seals atop the container and is configured to support the affinity column within. The lid can further include a vacuum port Vc for connection with an air tube connected to the vacuum source so that a vacuum can be drawn within the cup to facilitate flow of fluid through the affinity column. The cup can further include vial stand 2262 for supporting the vial 2221 beneath the affinity column. The cup can further include an interface plug 2263 for sealing atop the affinity column and having ports p1, p2, p3 for passage of the fluid sample from the binding/mixing reservoir (through p1) and for injection of the respective washer and elution buffers (p2, p3). FIGS. 25A-25C show a mold tool 2270 having pins 2271 by which the plug 2263 can be injection molded.

In one aspect, the methods described herein can be performed by a partly or fully automated process. In some embodiments, the process can utilize one or more cassettes or cartridges that hold the sample during at least a portion of the process. In some embodiments, the process is a fully automated of a cassette based process. For full automation, all solutions will be delivered from reservoirs. In some embodiments, the setup utilizes one or more valves that are controlled electronically or all the valves can be controlled automatically. In some embodiments, the collection cup (also referred to as a “cassette”) is a consumable that is used for each sample and disposed thereafter. In some embodiments, the final sample vial that obtains the sample concentrated by the process may still be handled separately. In some embodiments, the process by which a sample can be obtained, treated and analyzed can include any of the following parts.

Part 1. In a first aspect, the process can include any of: obtaining the sample, logging the sample, adding an extraction buffer, and stomaching the sample for extraction. In some embodiments, if the overlay is charged with dirt and/or heavy solids, the sample can be subjected to low-speed centrifugation and/or the extract decanted. This is optional and is only rarely required due to some processing of produce having substantial debris (e.g. spinach). Next, the sample is concentrated by filtration, for example, by using filter aid materials as described herein which can utilize any of the process steps described herein. In some embodiments, the extract is concentrated by filtration through a filter cup (e.g. 0.45 micron filter cup) and retained in a filter aid material (e.g. filter cake), which retains the extract and target of interest (e.g. bacteria, pathogen, etc) as described previously.

Part 2. In a second aspect, a flow-through process can be utilized, which can employ flow-through systems such as that described above in FIG. 22, which can be manually controlled, or can be partly or fully automated. In some embodiments, the filter cup 2250 having the concentrated sample is transferred to a top of the flow-through system. A lysis buffer (e.g. 2-7 mls) is added, which optionally can be heated. In some embodiments, a heating source 2202 (e.g. incandescent bulb, heater, etc.) can be used to maintain an elevated temperature for a period of time (e.g. at least 5 minutes, 5-10 minutes, 10 minutes or more). A valve V1 can then be opened on a conduit C1 that connects to a binding/mixing vessel 2214 (e.g. syringe barrel). One or more vacuum valves are opened (Va, Vb) to apply vacuum to the binding/mixing vessel to draw the lysed bacteria solution into the collection vessel. The vacuum valve (Vb) is then closed. A suitable amount (e.g. 3-7 mls) of neutralizing or binding buffer from buffer reservoir 2215 is then added to the lysed bacteria solution in binding/mixing vessel. In some embodiments, the solution is then agitated, for example by operating agitator motor 230 for a suitable period of time (e.g. at least 5 seconds, 5-15 seconds). Next, valve V3 is opened that connects to a DNA affinity column 2216. A vacuum can be connected to the collection cup chamber. Opening vacuum valve Vc applies vacuum to the collection cup 260 and draws the neutralizer sample solution through the DNA affinity column 2216. In some embodiments, a wash buffer is injected into the DNA column from wash buffer reservoir 2218 (e.g. syringe) to rinse through the column. The vacuum remains open to draw the liquid through the column. In some embodiments, a second round of wash buffer is added to rinse through the column in the same manner. When all fluid is run through the DNA column, a collection vial is positioned beneath the DNA column. A suitable amount (75-100 microliters) of elution buffer is introduced into the DNA affinity column from elution buffer reservoir 2219 (e.g., syringe). Vacuum then suctions the liquid through the DNA affinity column for collection in the sample vial 2221 in the collection cup 2260. The vacuum can then be shut down and the vial containing the sample solution of target DNA (if any target is present) can be closed and removed.

Part 3. In a third aspect, this process occurs after the flow-through setup process detailed above. In some embodiments, small aliquots from the vial are added to PCR reactions. The results of the PCR reactions are used to assess a presence or absence of the target (e.g. assess pathogen risk) and/or determine levels of bacteria of interest (e.g. pathogen or good bacteria). The analytical results can then be reported to the user.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). As used herein, including in the claims, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” For example, the articles “a” and “an” as used in this application and the appended claims should generally be construed to mean “one or more” unless specified otherwise or clear from the context to be directed to a singular form. Unless specifically stated otherwise, the term “some” refers to one or more. Moreover, the term “or” is intended to mean an inclusive “or” rather than an exclusive “or.” That is, unless specified otherwise or clear from the context, the phrase, for example, “X employs A or B” is intended to mean any of the natural inclusive permutations. That is, for example the phrase “X employs A or B” is satisfied by any of the following instances: X employs A; X employs B; or X employs both A and B. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

The various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

If implemented in hardware, an example hardware configuration may comprise a processing system in a wireless node. The processing system may be implemented with bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement the signal processing functions of the PHY layer. In the case of a user terminal 120 (see FIG. 1); a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer readable medium. Software shall be construed broadly to mean instructions, data, or any combination thereof, whether referred to as software, firmware, middleware, microcode, hardware description language, or otherwise. Computer-readable media include both computer storage media and communication media including any medium that facilitates transfer of a computer program from one place to another. The processor may be responsible for managing the bus and general processing, including the execution of software modules stored on the machine-readable storage media. A computer-readable storage medium may be coupled to a processor such that the processor can read information from, and write information to, the storage medium. In the alternative, the storage medium may be integral to the processor. By way of example, the machine-readable media may include a transmission line, a carrier wave modulated by data, and/or a computer readable storage medium with instructions stored thereon separate from the wireless node, all of which may be accessed by the processor through the bus interface. Alternatively, or in addition, the machine-readable media, or any portion thereof, may be integrated into the processor, such as the case may be with cache and/or general register files. Examples of machine-readable storage media may include, by way of example, RAM (Random Access Memory), flash memory, phase change memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module below, it will be understood that such functionality is implemented by the processor when executing instructions from that software module.

Also, any connection is properly termed a computer-readable medium. For example, if the software is transmitted from a website, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray® disc where disks usually reproduce data magnetically, while discs reproduce data optically with lasers. Thus, in some aspects computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media.

Thus, certain aspects may comprise a computer program product for performing the operations presented herein. For example, such a computer program product may comprise a computer-readable medium having instructions stored (and/or encoded) thereon, the instructions being executable by one or more processors to perform the operations described herein. For example, instructions for performing the operations described herein and illustrated in the appended figures.

Further, it should be appreciated that modules and/or other appropriate means for performing the methods and techniques described herein can be downloaded and/or otherwise obtained by a user terminal and/or base station as applicable. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes and variations may be made in the arrangement, operation and details of the methods and apparatus described above without departing from the scope of the claims.

1. A method of sampling for a target, the method comprising:
filtering a fluid sample through one or more filter aid materials, wherein at least some of a desired target, if present, disposed within the fluid sample remains within the one or more filter materials;
lysing the at least some of the desired target remaining within the one or more filter materials to release to release molecules from the one or more desired targets remaining within the one or more filter materials; and
recovering the release molecules in sufficient yield for testing detection of the one or more desired targets.

2-31. (canceled)

32. A sample processing system comprising:
a container or conduit;
a filter disposed within the container or conduit for filtering a fluid sample in preparation for testing of one or more targets within the fluid sample;
one or more filter aid materials; and
a lysing means for lysing one or more targets remaining within the one or more filter aid material and/or filter to release molecules from the one or more targets remaining within the one or more filter materials and/or filter.

33-53. (canceled)

54. A sample treatment system for treating a sample, the system comprising a plurality of elements including:
an extraction container for holding a sample medium and/one or more filter materials exposed to an item and/or surface being sampled;
an extraction buffer reservoir having an extraction buffer therein and fluidically coupled to and upstream of the extraction container;
a filtration module fluidically coupled to and downstream of the extraction container, the filtration module having one or more filtering features therein;
an in situ lysis buffer reservoir having an in situ lysis buffer therein and fluidically coupled to the filtration module;
a binding/mixing vessel downstream of the extraction container;
an binding buffer reservoir having an binding buffer therein and fluidically coupled to the binding/mixing vessel;
a nucleic acid affinity column fluidically coupled to an downstream of the filtration system, wherein the system is configured so that a purified and concentrated sample is collected downstream of the nucleic acid affinity column; and
a washing buffer reservoir having a washer buffer therein and an elution buffer reservoir having an elution buffer therein, each fluidically coupled to the filtration module.

55-63. (canceled)

64. A method of treating sample, the method comprising:
extracting a fluid sample from a sample medium and/or filter aid materials disposed in an extraction bag or container by use of an extraction buffer;
transporting the extracted fluid sample to a filtration module and lysing by use of a lysis buffer;
transporting the lysed filtered sample to a binding/mixing vessel and mixing with a binding buffer;
transporting the mixed fluid sample to nucleic acid affinity column and adding a washing buffer; and
eluting the fluid sample in the affinity column by use of a washing buffer and an elution buffer, thereby producing a purified, concentrated fluid sample for testing.

65. The method of claim 64, wherein the purified, concentrated fluid sample is suitable for testing without requiring enrichment.

66. The method of claim 64, further comprising:
adding enrichment media to the filter aid materials after lysing in situ with the lysis buffer and maintaining a suitable temperature for a short enrichment within the filter aid materials.

67. The method of claim 64, further comprising:
extracting the concentrated sample from the filter aid materials into a warm enrichment media and maintaining at a suitable temperature for a short enrichment of the extracted sample solution.

68. The method of claim 64, wherein the purified, concentrated fluid sample is concentrated by filtration in filter aid materials without lysing in situ within the filter aid materials and adding an enrichment media to the concentrated sample and maintaining at a suitable temperature for enrichment of any DNA therein to reach suitable levels for testing.

69. The method of claim 64, wherein each of the filtration unit, binding/mixing vessel, and nucleic acid affinity column are fluidically coupled to each other and to an associated buffer reservoir and a waste receptacle through an interconnected network of fluid conduits.

70. The method of claim 69, wherein the interconnected network of fluid conduits includes a plurality of valves and one or more vents configured so that the fluid sample flows through the entire system without requiring any mechanical transport.

71. The method of claim 70, wherein the system is configured so that the fluid sample flows through the entire interconnected network by use of a single vacuum source.

72. The method of claim 70, is configured so that the fluid sample flows through the entire system to produce the purified, concentrate fluid sample within two hours or less.

73. The method of claim 64, wherein the flow of sample through the system is controlled by a centralized control unit.

74. A method of treating a sample, the method comprising:
placing a sample medium and/or filter aid materials in an extraction bag and fluidically coupling the extraction bag to a sample treatment system;
fluidically coupling a removable sample treatment cartridge within the sample treatment system, the cartridge including a filtration unit, binding/mixing vessel, and nucleic acid affinity column, and the system including associated buffer reservoirs and a waste receptacle, wherein together, the cartridge and system define a network of fluidic conduits and valves;
operating the system by use of one or more vacuum sources and/or injection units so as to control facilitate controlled flow of fluid sample, buffers and any waste product through the network of fluidic conduits, thereby:
extracting a fluid sample from a sample medium and/or filter aid materials disposed in an extraction bag or container by use of an extraction buffer,
transporting the extracted fluid sample to a filtration module and lysing by use of an in situ lysis buffer,
transporting the lysed filtered sample to a binding/mixing vessel and mixing with a binding buffer,
transporting the mixed fluid sample to nucleic acid affinity column and adding a washing buffer, and
eluting the fluid sample in the affinity column by use of an elution buffer so as to purify and concentrate the fluid sample; and
dispensing the producing a purified, concentrated fluid sample from the cartridge into a sample test tube or container for testing.

75. The method of claim 74, further comprising:
fluidically coupling a removable sample injection unit that includes tubing that connects the extraction bag to the cartridge and an extraction buffer reservoir.

76. The method of claim 74, wherein the interconnected network of fluid conduits includes a plurality of valves and one or more vents configured so that the fluid sample flows through the entire system without requiring any mechanical transport.

77. The method of claim 76, further comprising controlling flow of fluid sample through the interconnected network by applying a vacuum and actuating the plurality of valves by a centralized control unit.

78. The method of claim 77, is configured so that the fluid sample flows through the entire system by use of a single vacuum source.

79. The method of claim 76, wherein the fluidic conduits of the cartridge comprise flexible tubing and the system includes an interface having pinch valves that engages the flexible tubing.

80. The method of claim 76, wherein the system is configured so that the fluid sample flows through the entire system to produce the purified, concentrate fluid sample within two hours or less.

81. A method of treating a sample, the method comprising:
filtering a fluid sample with a sample cup having filter aid materials therein, thereby trapping target molecules from the fluid sample in the filter aid materials and discarding waste water from filtering the fluid sample;
placing the sample cup having the filter aid materials in a sample treatment system;
fluidically coupling the sample cup with a binding/mixing vessel, nucleic acid affinity column, associated buffer reservoirs and a waste receptacle of the system, wherein the system further includes a network of fluidic conduits and one or more valves that facilitate controlled fluid flow through the system;
operating the system by use of one or more vacuum sources and actuation of the one or more valves so as to facilitate controlled flow of the fluid sample, buffers and any waste product through the network of fluidic conduits, thereby:
lysing the target molecules within the filter aid materials disposed in the sample cup by use of an in situ lysate buffer introduced into the sample cup by the system so as to create a lysed fluid sample having DNA released from the target molecules;
transporting the lysed fluid sample containing the released DNA, by application of vacuum, from the sample cup into the binding/mixing vessel and mixing with a washing buffer and/or binding buffer;
transporting, by selective actuation of the one or more valves, the mixed fluid sample containing the released DNA into an nucleic acid affinity column and adding an elution buffer, and
eluting, by selective actuation of the one or more valves, a concentrated fluid sample containing the DNA from the target molecules from the column into a test tube.

82. The method of claim 81, wherein a portion of the system including the binding/mixing reservoir and the nucleic acid affinity column comprises a removable/replaceable cartridge.

83. The method of claim 81, wherein the sample cup is a standard, off-the-shelf sample cup.

84-100. (canceled)
